(19) United States
(12) Patent Application Publication
(10) Pub. No.: US 2023/0405001 A1
(43) Pub. Date: Dec. 21, 2023

(54) TREATMENT OR PREVENTION OF HIV INFECTION

(71) Applicant: Janssen Sciences Ireland Unlimited Company, Co Cork (IE)

(72) Inventors: Steven M. Vesole, Redwood City, CA (US); Guenter Karl Wilhelm Kraus, Frankfurt AM Main (DE); Herta Maria Ludovica Crauwels, Wilrijk (BE); René Holm, Odense (DK); Nico Rudolph Niemeijer, Sint-Oedenrode (NL); Iwan Caroline F. Vervoort, Hoogstraten (BE)

(21) Appl. No.: 18/037,204

(22) PCT Filed: Nov. 17, 2021
(86) PCT No.: PCT/US2021/072453

§ 371 Date: May 16, 2023

Related U.S. Application Data
(60) Provisional application No. 63/114,997, filed on Nov. 17, 2020.

(51) Int. Cl. A61K 31/505; A61K 9/00; A61K 38/47; A61K 9/50

(52) U.S. Cl. CPC A61K 31/505; A61K 9/0019; A61K 38/47; A61K 9/5031

(57) ABSTRACT

The present invention relates to the treatment or prevention of HIV infection using rilpivirine or a pharmaceutically acceptable salt thereof in the form of micro- or nanoparticles in suspension in combination with a hyaluronidase. The present invention also relates to rilpivirine or a pharmaceutically acceptable salt thereof in the form of micro- or nanoparticles in suspension.

### TECHNICAL FIELD

The present invention relates to the treatment or prevention of HIV infection using rilpivirine or a pharmaceutically acceptable salt thereof in the form of micro- or nanoparticles in suspension in combination with a hyaluronidase. The present invention also relates to rilpivirine or a pharmaceutically acceptable salt thereof in the form of micro- or nanoparticles in suspension.

### BACKGROUND AND RELATED ART

The treatment of human immunodeficiency virus (HIV) infection, known as the cause of the acquired immunodeficiency syndrome (AIDS), remains a major medical challenge. HIV is able to evade immunological pressure, to adapt to a variety of cell types and growth conditions and to develop resistance against anti-HIV drugs. The latter include nucleoside reverse transcriptase inhibitors (NRTIs), non-nucleoside reverse transcriptase inhibitors (NNRTIs), nucleotide reverse transcriptase inhibitors (NtRTIs), HIV-protease inhibitors (PIs), integrase strand transfer inhibitors (INSTIs) and HIV fusion inhibitors.

Although effective in suppressing HIV infection, each of these drugs, when used alone, is confronted with the emergence of resistant mutants. This led to the introduction of combination therapy of several anti-HIV agents usually having a different activity profile. In particular the introduction of “HAART” (highly active anti-retroviral therapy) resulted in a remarkable improvement in anti-HIV therapy, leading to a dramatic reduction in HIV-associated morbidity and mortality. Current guidelines for antiretroviral therapy recommend dual or triple combination therapy regimens. However, none of the currently available drug therapies is capable of completely eradicating HIV infection. Even HAART can face the emergence of resistance, often due to non-adherence and non-persistence with antiretroviral therapy. In these cases HAART can be made effective again by replacing one of its components by one of another class. If applied correctly, treatment with HAART combinations can suppress the virus for many years, up to decades, to a level where it no longer can cause the outbreak of AIDS.

As HIV infection can currently not be completely eradicated, persons infected with HIV pose a potential risk of infecting others. People may live for years with the infection without experiencing any effects of it and therefore may be unaware of the risk of further transferring the virus to others. Prevention of HIV transmission therefore is crucial. Prevention currently focuses on avoiding transmission by sexual contacts, in particular by the use of condoms in populations at risk of being infected, on careful monitoring of blood samples for the presence of HIV and on avoiding of contact with blood of potentially infected subjects.

Despite these measures there is always an imminent risk of individuals being in contact with HIV infected persons and becoming infected. This in particular is the case for those providing medical care to infected patients or patients at risk of being infected such as physicians, nurses or dentists. Another group of individuals at risk are breast-fed infants whose mother is infected or at risk of becoming infected, especially in developing countries where alternatives for breast-feeding are less obvious.

Currently available oral therapies require at least once daily dosing. Hence people living with HIV are reminded on a daily basis of their HIV-positive status and daily dosing may also lead to disclosure of their HIV positive status. Daily dosing requires storage and transport of a large number or volume of pills and there remains the risk of patients forgetting to take their daily dose, thereby failing to comply with the prescribed dosage regimen. As well as reducing the effectiveness of the treatment, this also leads to the emergence of viral resistance.

One class of HIV drugs often used in HAART is the NNRTIs. Rilpivirine is an anti-retroviral of the NNRTI class that is used for the treatment of HIV infection. Rilpivirine is a second-generation NNRTI with higher potency and a reduced side effect profile compared with older NNRTIs. Rilpivirine activity is mediated by non-competitive inhibition of HIV-1 reverse transcriptase.

Rilpivirine not only shows pronounced activity against wild type HIV, but also against many of its mutated variants. Rilpivirine, its pharmacological activity, as well as a number of procedures for its preparation have been described in WO 03/16306.

Rilpivirine has been approved for the treatment of HIV infection and is commercially available as a single agent tablet (EDURANT®) containing 25 mg of rilpivirine base equivalent per tablet for once-daily oral administration as well as single tablet regimens for once-daily oral administration (COMPLERA®, ODEFSEY®, JULUCA®).

WO2007147882 discloses intramuscular or subcutaneous injection of a therapeutically effective amount of rilpivirine in micro- or nanoparticle form, having a surface modifier adsorbed to the surface thereof; and a pharmaceutically acceptable aqueous carrier; wherein the rilpivirine active ingredient is suspended. Products comprising rilpivirine for the treatment of HIV infection by injection once monthly or every two months are currently in development.

A prolonged release suspension for injection of rilpivirine for administration in combination with a prolonged release suspension for injection of cabotegravir has been approved in Canada as CABENUVA® and the EMA has recommended the granting of the marketing authorisation for a prolonged-release suspension for injection of rilpivirine (REKAMBYS®) in Europe. These are the first anti-retrovirals to be provided in a long-acting injectable formulation for administration at intervals of greater than one day.

For drugs administered by subcutaneous or intramuscular injection, such as rilpivirine, patient tolerability is an additional concern, certainly when larger volumes are injected. For example, administration by subcutaneous or intramuscular injection can result in irritation, inflammation, swelling, acute pain and/or redness and bruising during and after injection at the injection site (injection site reactions). Subcutaneous and intramuscular injections, certainly when larger volumes are injected, may also be associated with the manifestation of a bump at the surface of the skin at the injection site. Such effects are generally exaggerated by a high injection volume. Such a bump may reveal that the subject concerned received a high volume injection and may hence reveal the HIV positive status of the subject.

Therefore, in addition to the need to provide an effective method for preventing HIV transmission or treating HIV infection which requires infrequent dosing, i.e. dosing only once every few months or longer, there is also a need for this method to be well tolerated, which in turns improves patient compliance. There is also a need for this method to be non-visible to the outside world.

### SUMMARY OF THE INVENTION

In a first aspect there is provided a method for the treatment or prevention of HIV infection in a subject in need thereof, the method comprising administering to the subject a therapeutically effective amount of rilpivirine or a pharmaceutically acceptable salt thereof in the form of micro- or nanoparticles in suspension by intramuscular injection or subcutaneous injection, wherein the rilpivirine or a pharmaceutically acceptable salt thereof is administered in combination with a hyaluronidase that is administered by intramuscular injection or subcutaneous injection, and wherein the rilpivirine or pharmaceutically acceptable salt thereof and the hyaluronidase are administered intermittently at a time interval of about three months to about two years.

In a second aspect there is provided rilpivirine or a pharmaceutically acceptable salt thereof and a hyaluronidase for use in the treatment or prevention of HIV infection in a subject, wherein the rilpivirine or pharmaceutically acceptable salt thereof is in the form of micro- or nanoparticles in suspension, wherein the rilpivirine or pharmaceutically acceptable salt thereof and the hyaluronidase are administered to the subject by intramuscular injection or subcutaneous injection, and wherein the rilpivirine or pharmaceutically acceptable salt thereof and the hyaluronidase are administered intermittently at a time interval of about three months to about two years.

In a third aspect there is provided products containing rilpivirine or a pharmaceutically acceptable salt thereof and a hyaluronidase as a combined preparation for simultaneous or sequential use in the treatment or prevention of HIV infection by intramuscular injection or subcutaneous injection, wherein the rilpivirine or pharmaceutically acceptable salt thereof is in the form of micro- or nanoparticles in suspension, and wherein the rilpivirine or pharmaceutically acceptable salt thereof and the hyaluronidase are administered intermittently at a time interval of about three months to about two years.

In a fourth aspect there is provided a kit of parts comprising rilpivirine or a pharmaceutically acceptable salt thereof and a hyaluronidase for simultaneous or sequential use in the treatment or prevention of HIV infection by intramuscular injection or subcutaneous injection, wherein the rilpivirine or a pharmaceutically acceptable salt thereof is in the form of micro- or nanoparticles in suspension, and wherein the rilpivirine or pharmaceutically acceptable salt thereof and the hyaluronidase are administered intermittently at a time interval of about three months to about two years.

In a fifth aspect there is provided rilpivirine or a pharmaceutically acceptable salt thereof in the form of a suspension of micro- or nanoparticles for use in the treatment or prevention of HIV infection by intramuscular injection or subcutaneous injection, wherein the rilpivirine or pharmaceutically acceptable salt thereof is administered in combination with a hyaluronidase that is administered by intramuscular injection or subcutaneous injection, and wherein the rilpivirine or pharmaceutically acceptable salt thereof and the hyaluronidase are administered intermittently at a time interval of about three months to about two years.

In a sixth aspect there is provided use of rilpivirine or a pharmaceutically acceptable salt thereof for the manufacture of a medicament for treating or preventing HIV infection in a subject, wherein the rilpivirine or pharmaceutically acceptable salt thereof is in the form of micro- or nanoparticles in suspension and is administered in combination with a hyaluronidase, wherein the rilpivirine or pharmaceutically acceptable salt thereof and the hyaluronidase are administered to the subject by intramuscular injection or subcutaneous injection, and wherein the rilpivirine or pharmaceutically acceptable salt thereof and the hyaluronidase are administered intermittently at a time interval of about three months to about two years.

In a seventh aspect there is provided a combination comprising rilpivirine or a pharmaceutically acceptable salt thereof and a hyaluronidase, wherein the rilpivirine or a pharmaceutically acceptable salt thereof is in the form of micro- or nanoparticles in suspension.

In an eighth aspect there is provided a kit of parts comprising rilpivirine or a pharmaceutically acceptable salt thereof and a hyaluronidase, wherein the rilpivirine or a pharmaceutically acceptable salt thereof is in the form of micro- or nanoparticles in suspension.

Administration of rilpivirine in combination with a hyaluronidase by subcutaneous or intramuscular injection improves patient tolerability compared with subcutaneous or intramuscular injection administration of rilpivirine alone, in particular when large volumes are injected. The hyaluronidase may facilitate a more rapid administration of the rilpivirine as it may lower the resistance of the tissue against which the rilpivirine suspension is delivered. The hyaluronidase may reduce leakage of the rilpivirine from the site of injection by decreasing the tissue backpressure. The hyaluronidase may also allow for delivery of larger volumes in patients with less subcutaneous tissue (or lower body mass index). The hyaluronidase may allow the use of a shorter needle.

In addition, it has surprisingly been found that the extended, sustained or prolonged release of rilpivirine into the blood plasma achieved by intramuscular injection or subcutaneous injection of a suspension of rilpivirine micro- or nanoparticles can be maintained when rilpivirine is administered with a hyaluronidase as defined herein. As discussed in more detail below in the section titled “Hyaluronidase”, hyaluronidases are used for increasing the dispersion and absorption of injected active pharmaceutical ingredients. In view of this, it is surprising that the inventors have demonstrated that administration of a hyaluronidase with rilpivirine maintains an extended, sustained or prolonged release of rilpivirine into the bloodstream.

In a ninth aspect there is provided rilpivirine or a pharmaceutically acceptable salt thereof in the form of micro- or nanoparticles in suspension, wherein the micro- or nanoparticles have a Dᵥ90 of from about 1 μm to about 10 μm.

In a tenth aspect there is provided a pharmaceutical composition comprising the rilpivirine or a pharmaceutically acceptable salt thereof as defined in the ninth aspect.

In an eleventh aspect there is provided the rilpivirine or a pharmaceutically acceptable salt thereof as defined in the ninth aspect for use in the treatment or prevention of HIV infection in a subject.

In a twelfth aspect there is provided a method for treating or preventing HIV infection in a subject, the method comprising administering rilpivirine or a pharmaceutically acceptable salt thereof according to the ninth aspect of the invention, i.e. in the form of micro- or nanoparticles in suspension, wherein the micro- or nanoparticles have a Dᵥ90 of from about 1 μm to about 10 μm, to the subject.

In a thirteenth aspect there is provided use of rilpivirine or a pharmaceutically acceptable salt thereof according to the ninth aspect of the invention, i.e. in the form of micro- or nanoparticles in suspension, wherein the micro- or nanoparticles have a Dᵥ90 of from about 1 μm to about 10 μm, for the manufacture of a medicament for treating or preventing HIV infection in a subject.

Rilpivirine in the form of micro- or nanoparticles having a Dᵥ90 of from about 1 μm to about has surprisingly been found to lower, i.e. flatten, the dissolution profile of rilpivirine compared to rilpivirine in the form of micro- or nanoparticles having a lower Dᵥ90. Thus, administration of rilpivirine in the form of micro- or nanoparticles having a Dᵥ90 of from about 1 μm to about 10 μm modulates rilpivirine exposure to flatten, i.e. lower the Cmax of, the pharmacokinetic curve while maintaining sustained or prolonged release of rilpivirine into the blood plasma. Administration of rilpivirine in the form of micro- or nanoparticles having a Dᵥ90 of from about 1 μm to about 10 μm may result in an improved peak-trough ratio at multiple doses compared to administration of rilpivirine in the form of micro- or nanoparticles having a lower Dᵥ90.

### BRIEF DESCRIPTION OF THE FIGURES

The invention will be described, by way of example only, with reference to the accompanying figures.

FIG. 1: Mean plasma concentration over time following administration of a rilpivirine nanosuspension and hyaluronidase according to the invention and of a rilpivirine nanosuspension alone.

FIG. 2: Mean plasma concentration over six months following administration of a rilpivirine suspension and hyaluronidase according to the invention and of a rilpivirine suspension alone.

FIG. 3: Dissolution studies with rilpivirine suspensions of varying particle size

FIG. 4: Further dissolution studies with rilpivirine suspensions of varying particle size

These figures are explained further in the “Examples” section.

### DISCLOSURE OF THE INVENTION

This application has been drafted in sections to aid readability. However, this does not mean that each section is to be read in isolation. To the contrary, unless otherwise specified, each section is to be read with cross-referencing to the other sections, i.e. taking the entire application as a whole. No artificial separation of embodiments is intended, unless explicitly stated.

Thus, all of the embodiments described herein relating to the first aspect of the invention apply equally to, i.e. are also disclosed in relation to/combination with aspects two to eight described herein. Also, all of the embodiments described herein relating to the ninth aspect of the invention apply equally to, i.e. are also disclosed in relation to/combination with, the tenth to thirteenth aspects of the invention.

### DETAILED DESCRIPTION OF THE INVENTION

Rilpivirine

Rilpivirine (4-[[4-[[4-[(1E)-2-cyanoethenyl]-2,6-dimethylphenyl]amino]-2-pyrimidinyl]amino]benzonitrile; TMC278) has the following structural formula:

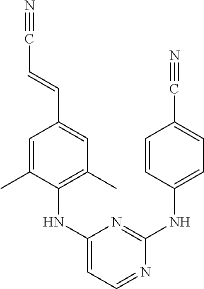 [SMILES: Cc1cc(/C=C/C#N)cc(C)c1Nc1ccnc(Nc2ccc(C#N)cc2)n1]

By “rilpivirine” it is meant rilpivirine having the structural formula shown above, i.e. the free base form.

The rilpivirine or a pharmaceutically acceptable salt thereof as used in the first and ninth aspects of the invention is in the form of micro- or nanoparticles in suspension, i.e. microparticles or nanoparticles of the rilpivirine or a pharmaceutically acceptable salt thereof in a suspension, in particular micro- or nanoparticles of the rilpivirine or a pharmaceutically acceptable salt thereof suspended in a pharmaceutically acceptable carrier, such as for example a pharmaceutically acceptable aqueous carrier.

Pharmaceutically acceptable salts of rilpivirine means those where the counterion is pharmaceutically acceptable. The pharmaceutically acceptable salts are meant to comprise the therapeutically active non-toxic acid addition salt forms which rilpivirine is able to form. These salt forms can conveniently be obtained by treating rilpivirine with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfamic, 2-hydroxybenzoic, 4-amino-2-hydroxybenzoic and the like acids.

In a preferred embodiment of the first and ninth aspects of the invention the rilpivirine or a pharmaceutically acceptable salt thereof used in the invention is rilpivirine, i.e. rilpivirine in its free base form.

The skilled person would understand that the size of the micro- or nanoparticles in the first aspect of the invention should be below a maximum size above which administration by subcutaneous or intramuscular injection becomes impaired or even is no longer possible. The maximum size depends for example on the limitations imposed by the needle diameter or by adverse reactions of the body to large particles, or both.

In a preferred embodiment of the first aspect of the invention, the rilpivirine or a pharmaceutically acceptable salt thereof is in the form of nanoparticles.

In an embodiment of the first aspect of the invention, the micro- or nanoparticles described herein have an average effective particle size of less than about 20 μm. In an embodiment of the first aspect of the invention the micro- or nanoparticles have an average effective particle size of less than about 10 μm. In an embodiment of the first aspect of the invention, the micro- or nanoparticles have an average effective particle size of less than about 5 μm. In an embodiment of the first aspect of the invention, the micro- or nanoparticles have an average effective particle size of less than about 1 μm. In an embodiment of the first aspect of the invention, the micro- or nanoparticles have an average effective particle size of less than about 500 nm.

In another embodiment of the first aspect of the invention, the micro- or nanoparticles described herein have an average effective particle size of from about 25 nm to about 20 μm. In another embodiment of the first aspect of the invention, the micro- or nanoparticles have an average effective particle size of from about 25 nm to about 10 μm (e.g. about 200 nm to about 10 μm). In another embodiment of the first aspect of the invention, the micro- or nanoparticles have an average effective particle size of from about 25 nm to about 5 μm (e.g. about 200 nm to about 5 μm). In another embodiment of the first aspect of the invention, the micro- or nanoparticles have an average effective particle size of from about nm to about 1 μm. In another embodiment of the first aspect of the invention, the micro- or nanoparticles have an average effective particle size of from about 25 nm to about 500 nm.

In a preferred embodiment of the first aspect of the invention, the micro- or nanoparticles described herein have an average effective particle size of from about 100 nm to about 300 nm. In another preferred embodiment of the first aspect of the invention, the micro- or nanoparticles have an average effective particle size of from about 150 nm to about 250 nm. In a particularly preferred embodiment of the first aspect of the invention the micro- or nanoparticles have an average effective particle size of about 180 nm to about 220 nm, e.g. about 200 nm.

In an alternative embodiment of the first and ninth aspects of the invention, the micro- or nanoparticles have an average effective particle size of from about 0.2 μm to about 3 μm. In an embodiment of the first and ninth aspects of the invention, the micro- or nanoparticles have an average effective particle size of from about 0.4 μm to about 3 μm. In an embodiment of the first and ninth aspects of the invention, the micro- or nanoparticles have an average effective particle size of from about 0.6 μm to about 3 μm. In an embodiment of the first and ninth aspects of the invention, the micro- or nanoparticles have an average effective particle size of from about 0.7 μm to about 3 μm. In an embodiment of the first and ninth aspects of the invention, the micro- or nanoparticles have an average effective particle size of from about 0.8 μm to about 3 μm. In an embodiment of the first and ninth aspects of the invention, the micro- or nanoparticles have an average effective particle size of from about 0.9 μm to about 3 μm. In an embodiment of the first and ninth aspects of the invention, the micro- or nanoparticles have an average effective particle size of from about 1 μm to about 3 μm. In an embodiment of the first and ninth aspects of the invention, the micro- or nanoparticles have an average effective particle size of from about 1 μm to about 2.5 μm. In an embodiment of the first and ninth aspects of the invention, the micro- or nanoparticles have an average effective particle size of from about 1 μm to about 2 μm. In an embodiment of the first and ninth aspects of the invention, the micro- or nanoparticles have an average effective particle size of 0.3 μm, of 0.4 μm, of 0.5 μm, of of 0.7 μm, of 0.8 μm, of 0.9 μm, of 1 μm, of 1.1 μm, of 1.2 μm, of 1.3 μm, of 1.4 μm, of 1.5 μm, of 1.6 μm, of 1.7 μm, of 1.8 μm, of 1.9 μm, of 2 μm, of 2.1 μm, of 2.2 μm, of 2.3 μm, of 2.4 μm, of 2.5 μm, of 2.6 μm, of 2.7 μm, of 2.8 μm, of 2.9 μm or of 3 μm, or of any sub-range or single value between 0.2 μm and 3 μm.

In an embodiment of the first and ninth aspects of the invention, the micro- or nanoparticles have an average effective particle size of from about 1.5 μm to about 3 μm. In an embodiment of the first and ninth aspects of the invention, the micro- or nanoparticles have an average effective particle size of from about 2 μm to about 3 μm, e.g. about 2.5 μm or about 2.7 μm.

The term “average effective particle size” as used herein refers to the volume-based median particle diameter (Dᵥ50), i.e. the diameter below which 50% by volume of the particle population is found.

In an alternative embodiment of the first aspect of the invention, the micro- or nanoparticles have a Dᵥ90 of from about 1 μm to about 10 μm.

The micro- or nanoparticles of the ninth aspect of the invention have a Dᵥ90 of from about 1 μm to about 10 μm.

In an embodiment of the first and ninth aspects of the invention, the micro- or nanoparticles have a Dᵥ90 of from about 1 μm to about 7 μm. In an embodiment of the first and ninth aspects, the micro- or nanoparticles have a Dᵥ90 of from about 1.5 μm to about 7 μm. In an embodiment of the first and ninth aspects, the micro- or nanoparticles have a Dᵥ90 of from about 2 μm to about 7 μm. In an embodiment of the first and ninth aspects, the micro- or nanoparticles have a Dᵥ90 of from about 2 μm to about 6 μm. In an embodiment of the first and ninth aspects, the micro- or nanoparticles have a Dᵥ90 of from about 2.5 μm to about 6.5 μm. In an embodiment of the first and ninth aspects, the micro- or nanoparticles have a Dᵥ90 of from about 2.5 μm to about 4 μm. In an embodiment of the first and ninth aspects, the micro- or nanoparticles have a Dᵥ90 of from about 3 μm to about 7 μm. In an embodiment of the first and ninth aspects, the micro- or nanoparticles have a Dᵥ90 of from about 4 μm to about 7 μm. In an embodiment of the first and ninth aspects, the micro- or nanoparticles have a Dᵥ90 of from about 3 μm to about 6 μm. In an embodiment, the micro- or nanoparticles have a Dᵥ90 of from about 3 μm to about 5.5 μm. In an embodiment of the first and ninth aspects, the micro- or nanoparticles have a Dᵥ90 of from about 4.5 μm to about 6.5 μm. In an embodiment of the first and ninth aspects, the micro- or nanoparticles have a Dᵥ90 of from about 5 μm to about 6 μm, e.g. about 5.5 μm. In an embodiment of the first and ninth aspects, the micro- or nanoparticles have a Dᵥ90 of 2 μm, of 2.1 μm, of 2.2 μm, of 2.3 μm, of 2.4 μm, of 2.5 μm, of 2.6 μm, of 2.7 μm, of 2.8 μm, of 2.9 μm, of 3 μm, of 3.1 μm, of 3.2 μm, of 3.3 μm, of 3.4 μm, of 3.5 μm, of 3.6 μm, of 3.7 μm, of 3.8 μm, of 3.9 μm, of 4 μm, of 4.1 μm, of 4.2 μm, of 4.3 μm, of 4.4 μm, of 4.5 μm, of 4.6 μm, of 4.7 μm, of 4.8 μm, of 4.9 μm, of 5 μm, of 5.1 μm, of 5.2 μm, of 5.3 μm, of 5.4 μm, of 5.5 μm, of 5.6 μm, of 5.7 μm, of 5.8 μm, of 5.9 μm, of 6 μm, of 6.1 μm, of 6.2 μm, of 6.3 μm, of 6.4 μm, of 6.5 μm, of 6.6 μm, of 6.7 μm, of 6.8 μm, of 6.9 μm, or of 7 μm, or of any sub-range or single value between 2 μm and 7 μm.

The term “Dᵥ90” as used herein refers to the diameter below which 90% by volume of the particle population is found.

In a particular embodiment of the first and ninth aspects, the micro- or nanoparticles have an average effective particle size (Dᵥ50) of from about 0.2 μm to about 3 μm and a Dᵥ90 of from about 1.8 μm to about 7 μm, or have an average effective particle size (Dᵥ50) of from about 0.6 μm to about 3 μm and a Dᵥ90 of from about 2.5 μm to about 6.5 μm, wherein for any embodiment the average effective particle size is lower than the Dᵥ90. In an alternative embodiment of the first and ninth aspects, the micro- or nanoparticles have an average effective particle size (Dᵥ50) of from about 0.6 μm to about 1.5 μm and a Dᵥ90 of from about 2.5 μm to about 4 μm, wherein for any embodiment the average effective particle size is lower than the Dᵥ90. In an alternative embodiment of the first and ninth aspects, the micro- or nanoparticles have an average effective particle size (Dᵥ50) of from about 1 μm to about 2 μm and a Dᵥ90 of from about 3.5 μm to about 5.5 μm, wherein for any embodiment the average effective particle size is lower than the Dᵥ90. In an alternative embodiment of the first and ninth aspects, the micro- or nanoparticles have an average effective particle size (Dᵥ50) of from about 2 μm to about 3 μm and a Dᵥ90 of from about 5.0 μm to about 6.5 μm, wherein for any embodiment the average effective particle size is lower than the Dᵥ90.

As can be seen from Example 3, administration of rilpivirine in the form of micro- or nanoparticles having a Dᵥ90 of from about 1 μm to about 10 μm has surprisingly been found to lower, i.e. flatten, the dissolution profile of rilpivirine. Thus, a particle size in this range modulates rilpivirine exposure to flatten, i.e. lower the Cmax of, the pharmacokinetic curve while maintaining sustained or prolonged release of rilpivirine into the blood plasma.

The average effective particle sizes, i.e. the volume-based median particle diameter (Dᵥ50), and the Dᵥ90 as used herein are determined by routine laser diffraction techniques, e.g. in accordance with ISO 13320:2009.

Laser diffraction relies on the principle that a particle will scatter light at an angle that varies depending on the size the particle and a collection of particles will produce a pattern of scattered light defined by intensity and angle that can be correlated to a particle size distribution. A number of laser diffraction instruments are commercially available for the rapid and reliable determination of particle size distributions. For example, particle size distribution may be measured by the conventional Malvern Mastersizer™ 3000 particle size analyzer from Malvern Instruments. The Malvern Mastersizer™ 3000 particle size analyzer operates by projecting a helium-neon gas laser beam through a transparent cell containing the particles of interest suspended in an aqueous solution. Light rays which strike the particles are scattered through angles which are inversely proportional to the particle size and a photodetector array measures the intensity of light at several predetermined angles and the measured intensities at different angles are processed by a computer using standard theoretical principles to determine the particle size distribution. Laser diffraction values may be obtained using a wet dispersion of the particles in distilled water.

Other methods that are commonly used in the art to measure volume-based median particle diameters (Dᵥ50) and Dᵥ90s include disc centrifugation, scanning electron microscope (SEM), sedimentation field flow fractionation and photon correlation spectroscopy.

In an embodiment of the first and ninth aspects of the invention, the micro- or nanoparticles have one or more surface modifiers adsorbed to their surface.

The surface modifier may be selected from known organic and inorganic pharmaceutical excipients, including various polymers, low molecular weight oligomers, natural products and surfactants. Particular surface modifiers that may be used in the invention include nonionic and anionic surfactants. Representative examples of surface modifiers include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives; polyoxyethylene stearates, colloidal silicon dioxide, sodium dodecylsulfate, carboxymethylcellulose sodium, bile salts such as sodium taurocholate, sodium desoxytaurocholate, sodium desoxycholate; methylcellulose, hydroxyethylcellulose, hydroxypropylcellulose, hydroxypropylmethylcellulose, magnesium aluminate silicate, polyvinyl alcohol (PVA), poloxamers, such as Pluronic™ F68, F108 and F127 which are block copolymers of ethylene oxide and propylene oxide; tyloxapol; Vitamin E-TGPS (α-tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters (Tweens™ 20, 40, 60 and 80); sorbitan esters of fatty acids (Span™ 20, 40, 60 and 80 or Arlacel™ 20, 40, 60 and 80); polyethylene glycols (such as those sold under the tradename Carbowax™ 3550 and 934); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP). If desired, two or more surface modifiers can be used in combination.

In an embodiment of the first and ninth aspects of the invention, the surface modifier is selected from a poloxamer, α-tocopheryl polyethylene glycol succinate, polyoxyethylene sorbitan fatty acid ester, and salts of negatively charged phospholipids or the acid form thereof. In a preferred embodiment of the first and ninth aspects of the invention, the surface modifier is selected from Pluronic™ F108, Vitamin E TGPS, Tween™ 80, and Lipoid™ EPG.

In an embodiment of the first and ninth aspects of the invention, the surface modifier is a poloxamer, in particular Pluronic™ F108. Pluronic™ F108 corresponds to poloxamer 338 and is the polyoxyethylene, polyoxypropylene block copolymer that conforms generally to the formula HO—[CH₂CH₂O]ₓ—[CH(CH₃)CH₂O]_{y}—[CH₂CH₂O]₂H in which the average values of x, y and z are respectively 128, 54 and 128. Other commercial names of poloxamer 338 are Hodag Nonionic™ 1108-F and Synperonic™ PE/F108. In one embodiment of the first and ninth aspects of the invention, the surface modifier comprises a combination of a polyoxyethylene sorbitan fatty acid ester and a phosphatidyl glycerol salt (in particular egg phosphatidyl glycerol sodium).

In an embodiment of the first and ninth aspects of the invention, the relative amount (w/w) of rilpivirine or a pharmaceutically acceptable salt thereof to the surface modifier is from about 1:2 to about 20:1, in particular from about 1:1 to about 10:1, e.g. from about 4:1 to about 6:1, preferably about 6:1.

In an embodiment of the first and ninth aspects of the invention, the micro- or nanoparticles of the invention comprise rilpivirine or a pharmaceutically acceptable salt thereof as defined herein and one or more surface modifiers as defined herein wherein the amount of rilpivirine or a pharmaceutically acceptable salt thereof is at least about 50% by weight of the micro- or nanoparticles, at least about 80% by weight of the micro- or nanoparticles, at least about 85% by weight of the micro- or nanoparticles, at least about 90% by weight of the micro- or nanoparticles, at least about 95% by weight of the micro- or nanoparticles, or at least about 99% by weight of the micro- or nanoparticles, in particular ranges between 80% and 90% by weight of the micro- or nanoparticles or ranges between 85% and 90% by weight of the micro- or nanoparticles.

In an embodiment of the first and ninth aspects of the invention, the suspension comprises a pharmaceutically acceptable aqueous carrier in which the rilpivirine or pharmaceutically acceptable salt thereof micro- or nanoparticles are suspended. The pharmaceutically acceptable aqueous carrier comprises sterile water, e.g. water for injection, optionally in admixture with other pharmaceutically acceptable ingredients. The latter comprise any ingredients for use in injectable formulations. These ingredients may be selected from one or more of a suspending agent, a buffer, a pH adjusting agent, a preservative, an isotonizing agent, a surface modifier, a chelating agent and the like ingredients. In one embodiment of the first and ninth aspects of the invention, said ingredients are selected from one or more of a suspending agent, a buffer, a pH adjusting agent, and optionally, a preservative and an isotonizing agent. Particular ingredients may function as two or more of these agents simultaneously, e.g. behave like a preservative and a buffer, or behave like a buffer and an isotonizing agent. In an embodiment of the first and ninth aspects of the invention said ingredients are selected from one or more of a buffer, a pH adjusting agent, an isotonizing agent, a chelating agent and a surface modifier. In an embodiment of the first and ninth aspects of the invention said ingredients are selected from one or more of a buffer, a pH adjusting agent, an isotonizing agent, and a chelating agent.

In an embodiment of the first and ninth aspects of the invention, the suspension additionally comprises a buffering agent and/or a pH adjusting agent. Suitable buffering agents and pH adjusting agents should be used in amount sufficient to render the dispersion neutral to very slightly basic (up to pH 8.5), preferably in the pH range of 7 to 7.5. Particular buffers are the salts of week acids. Buffering and pH adjusting agents that can be added may be selected from tartaric acid, maleic acid, glycine, sodium lactate/lactic acid, ascorbic acid, sodium citrates/citric acid, sodium acetate/acetic acid, sodium bicarbonate/carbonic acid, sodium succinate/succinic acid, sodium benzoate/benzoic acid, sodium phosphates, tris(hydroxymethyl)aminomethane, sodium bicarbonate/sodium carbonate, ammonium hydroxide, benzene sulfonic acid, benzoate sodium/acid, diethanolamine, glucono delta lactone, hydrochloric acid, hydrogen bromide, lysine, methanesulfonic acid, monoethanolamine, sodium hydroxide, tromethamine, gluconic, glyceric, gluratic, glutamic, ethylene diamine tetraacetic (EDTA), triethanolamine, including mixtures thereof. In an embodiment of the first and ninth aspects of the invention, the buffer is a sodium phosphate buffer, e.g. sodium dihydrogen phosphate monohydrate. In an embodiment the pH adjusting agent is sodium hydroxide.

In an embodiment of the first and ninth aspects of the invention, the suspension additionally comprises a preservative. Preservatives comprise antimicrobials and anti-oxidants which can be selected from the group consisting of benzoic acid, benzyl alcohol, butylated hydroxyanisole (BHA), butylated hydroxytoluene (BHT), chlorbutol, a gallate, a hydroxybenzoate, EDTA, phenol, chlorocresol, metacresol, benzethonium chloride, myristyl-γ-piccolinium chloride, phenylmercuric acetate and thimerosal. Radical scavengers include BHA, BHT, Vitamin E and ascorbyl palmitate, and mixtures thereof.

Oxygen scavengers include sodium ascorbate, sodium sulfite, L-cysteine, acetylcysteine, methionine, thioglycerol, acetone sodium bisulfite, isoacorbic acid, hydroxypropyl cyclodextrin. Chelating agents include sodium citrate, sodium EDTA, citric acid and malic acid. In an embodiment of the first and ninth aspects of the invention, the chelating agent is citric acid, e.g. citric acid monohydrate.

In an embodiment of the first and ninth aspects of the invention, the suspension additionally comprises an isotonizing agent. An isotonizing agent or isotonifier may be present to ensure isotonicity of the pharmaceutical compositions of the present invention, and includes sugars such as glucose, dextrose, sucrose, fructose, trehalose, lactose; polyhydric sugar alcohols, preferably trihydric or higher sugar alcohols, such as glycerin, erythritol, arabitol, xylitol, sorbitol and mannitol. Alternatively, sodium chloride, sodium sulfate, or other appropriate inorganic salts may be used to render the solutions isotonic. These isotonifiers can be used alone or in combination. The suspensions conveniently comprise from 0 to 10% (w/v), in particular 0 to 6% (w/v) of isotonizing agent. Of interest are nonionic isotonifiers, e.g. glucose, mannitol, as electrolytes may affect colloidal stability.

In an embodiment of the first aspect of the invention, each administration comprises up to about 600 mL of the suspension described herein, i.e. the volume of the suspension comprising the rilpivirine or a pharmaceutically acceptable salt thereof in the form of micro- or nanoparticles may have a volume of up to 600 mL. In an embodiment of the first aspect of the invention, each administration comprises from about 5 mL to about 600 mL of the suspension. In another embodiment of the first aspect of the invention, each administration comprises from about 5 mL to about 300 mL of the suspension. In another embodiment of the first aspect of the invention, each administration comprises from about mL to about 150 mL of the suspension. In another embodiment of the first aspect of the invention, each administration comprises from about 5 mL to about 25 mL of the suspension. In another embodiment of the first aspect of the invention, each administration comprises from about 6 mL to about 20 mL of the suspension. In another embodiment of the first aspect of the invention, each administration comprises from about 6 mL to about 18 mL of the suspension. In another embodiment of the first aspect of the invention, each administration comprises from about 6 mL to about 15 mL of the suspension. In another embodiment of the first aspect of the invention, each administration comprises from about 6 mL to about 12 mL of the suspension. In another embodiment of the first aspect of the invention, each administration comprises from about 9 mL to about 18 mL of the suspension. In another embodiment of the first aspect of the invention, each administration comprises from about 9 mL to about 15 mL of the suspension. In another embodiment of the first aspect of the invention, each administration comprises from about 9 mL to about 12 mL of the suspension. In another embodiment of the first aspect of the invention, each administration comprises about 6 mL of the suspension. In another embodiment of the first aspect of the invention, each administration comprises about 9 mL of the suspension. In another embodiment of the first aspect of the invention, each administration comprises about 12 mL of the suspension. In another embodiment of the first aspect of the invention, each administration comprises about 15 mL of the suspension. In another embodiment of the first aspect of the invention, each administration comprises about 18 mL of the suspension. In an embodiment of the first aspect of the invention, the rilpivirine suspension contains 300 mg rilpivirine/mL.

In an embodiment, the rilpivirine or pharmaceutically acceptable salt thereof of the first aspect of the invention (which is in the form of micro- or nanoparticles in suspension) is provided in a separate pharmaceutical composition from the hyaluronidase. As discussed further herein (e.g. in the section titled “Use of rilpivirine or a pharmaceutically acceptable salt thereof and hyaluronidase in the invention”), the separate pharmaceutical composition may be administered sequentially with a pharmaceutical composition comprising the hyaluronidase of the first aspect of the invention, or the separate pharmaceutical composition may be admixed with a pharmaceutical composition comprising the hyaluronidase of the invention prior to administration of the resulting admixed pharmaceutical composition.

In another embodiment, the rilpivirine or pharmaceutically acceptable salt thereof of the first aspect of the invention (which is in the form of micro- or nanoparticles in suspension) is provided in the same pharmaceutical composition as the hyaluronidase, i.e. the rilpivirine or pharmaceutically acceptable salt thereof is formulated in a combined pharmaceutical composition with the hyaluronidase.

In an embodiment of the first aspect of the invention, for the treatment of HIV infection, the dose to be administered may be calculated on a basis of about 300 mg to about 1200 mg/month, or about 450 mg to about 1200 mg/month, or about 450 mg to about 900 mg/month, or about 600 mg to about 900 mg/month, or about 450 mg to about 750 mg/month, or 450 mg/month, or 600 mg/month, or 750 mg/month, or 900 mg/month. Doses for other dosing regimens can readily be calculated by multiplying the monthly dose with the number of months between each administration. For example, in case of a dose of 450 mg/month, and in case of a time interval of 6 months between each administration, the dose to be administered in each administration is 2700 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment of the first aspect of the invention, for the treatment of HIV infection, the dose to be administered may be calculated on a basis of about 300 mg to about 1200 mg/4 weeks (28 days), or about 450 mg to about 1200 mg/4 weeks (28 days), or about 450 mg to about 900 mg/4 weeks (28 days), or about 600 mg to about 900 mg/4 weeks (28 days), or about 450 mg to about 750 mg/4 weeks (28 days) or 450 mg/4 weeks (28 days), or 600 mg/4 weeks (28 days), or 750 mg/4 weeks (28 days) or 900 mg/4 weeks (28 days). Doses for other dosing regimens can readily be calculated by multiplying the week or day dose with the number of weeks between each administration. For example, in case of a dose of 450 mg/4 weeks (28 days), and in case of a time interval of 24 weeks between each administration, the dose to be administered in each administration is 2700 mg. Or for example, in case of a dose of 750 mg/4 weeks (28 days), and in case of a time interval of 24 weeks between each administration, the dose to be administered in each administration is 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment of the first aspect of the invention, for the treatment of HIV infection, each administration of rilpivirine or a pharmaceutically acceptable salt thereof may comprise from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg or from about 2700 mg to about 4500 mg of the rilpivirine or pharmaceutically acceptable salt thereof.

Thus, the amount of the rilpivirine or pharmaceutically acceptable salt thereof in the pharmaceutical composition, i.e. the separate or combined pharmaceutical composition defined herein in relation to the first aspect of the invention, may be from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg or from about 2700 mg to about 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In the instance of prevention of HIV infection, each administration of rilpivirine or pharmaceutically acceptable salt thereof may comprise the same dosing as for therapeutic applications as described above.

In an embodiment of the first aspect of the invention, the rilpivirine or pharmaceutically acceptable salt thereof in the pharmaceutical composition, i.e. the separate or combined pharmaceutical composition defined herein, is used in an amount such that the blood plasma concentration of rilpivirine in the subject is kept at a level above about 12 ng/ml, preferably ranging from about 12 ng/ml to about 100 ng/ml, more preferably about 12 ng/ml to about 50 ng/ml for at least three months after administration, or at least 6 months after administration, or at least 9 months after administration, or at least 1 year after administration, or at least 2 years after each administration. In a preferred embodiment of the first aspect of the invention, the rilpivirine or pharmaceutically acceptable salt thereof in the pharmaceutical composition is used in an amount such that the blood plasma concentration of rilpivirine in the subject is kept at a level of from 12 ng/ml to 100 ng/ml for at least 6 months.

In a particular embodiment of the first aspect of the invention, the rilpivirine or pharmaceutically acceptable salt thereof is formulated and administered as micro- or nanoparticles in suspension wherein the formulation comprises the following components:
rilpivirine or a pharmaceutically acceptable salt thereof, in particular rilpivirine;
a surface modifier as defined herein, in particular poloxamer 338;
an isotonizing agent, in particular glucose monohydrate;
a buffer, in particular sodium dihydrogen phosphate;
a chelating agent, in particular citric acid monohydrate;
a pH adjusting agent, in particular sodium hydroxide; and
water, in particular water for injection.

In another particular embodiment of the first aspect of the invention, the rilpivirine or pharmaceutically acceptable salt thereof is formulated and administered as micro- or nanoparticles in suspension wherein the formulation comprises the following components:
rilpivirine or a pharmaceutically acceptable salt thereof, in particular rilpivirine;
poloxamer 338;
glucose monohydrate;
sodium dihydrogen phosphate;
citric acid monohydrate;
sodium hydroxide; and
water, in particular water for injection.

In one embodiment of the first and ninth aspects of the invention, the aqueous suspensions may comprise by weight, based on the total volume of the suspension:
(a) from 3% to 50% (w/v), or from 10% to 40% (w/v), or from 10% to 30% (w/v), of rilpivirine or a pharmaceutically acceptable salt thereof; in particular rilpivirine;
(b) from 0.5% to 10% (w/v), or from 0.5% to 5% (w/v), or from 0.5% to 2% (w/v) of a surface modifier; in particular poloxamer 338;
(c) from 0% to 10% (w/v), or from 0% to 5% (w/v), or from 0% to 2% (w/v), or from 0% to 1% (w/v) of one or more buffering agents; in particular sodium dihydrogen phosphate;
(d) from 0% to 10% (w/v), or from 0% to 6% (w/v), or from 0% to 5% (w/v), or from 0% to 3% (w/v), or from 0% to 2% (w/v) of an isotonizing agent; in particular glucose monohydrate;
(e) from 0% to 2% (w/v), or from 0% to 1% (w/v), or from 0% to 0.5% (w/v), or from 0% to 0.1% (w/v) of a pH adjusting agent; in particular sodium hydroxide;
(f) from 0% to 2% (w/v), or from 0% to 1% (w/v), or from 0% to 0.5% (w/v), or from 0% to 0.1% (w/v) of a chelating agent; in particular citric acid monohydrate;
(g) from 0% to 2% (w/v) preservatives; and
(h) water for injection q.s. ad 100%.

In one embodiment of the first and ninth aspects of the invention, the aqueous suspensions may comprise by weight, based on the total volume of the suspension:
(a) from 3% to 50% (w/v), or from 10% to 40% (w/v), or from 10% to 30% (w/v), of rilpivirine or a pharmaceutically acceptable salt thereof; in particular rilpivirine;
(b) from 0.5% to 10% (w/v), or from 0.5% to 5% (w/v), or from 0.5% to 2% (w/v) of a surface modifier; in particular poloxamer 338;
(c) from 0% to 10% (w/v), or from 0% to 5% (w/v), or from 0% to 2% (w/v), or from 0% to 1% (w/v) of one or more buffering agents; in particular sodium dihydrogen phosphate;
(d) from 0% to 10% (w/v), or from 0% to 6% (w/v), or from 0% to 5% (w/v), or from 0% to 3% (w/v), or from 0% to 2% (w/v) of an isotonizing agent; in particular glucose monohydrate;
(e) from 0% to 2% (w/v), or from 0% to 1% (w/v), or from 0% to 0.5% (w/v), or from 0% to 0.1% (w/v) of a pH adjusting agent; in particular sodium hydroxide;
(f) from 0% to 2% (w/v), or from 0% to 1% (w/v), or from 0% to 0.5% (w/v), or from 0% to 0.1% (w/v) of a chelating agent; in particular citric acid monohydrate; and
(g) water for injection q.s. ad 100%.

In a particular embodiment of the first aspect of the invention, the rilpivirine or pharmaceutically acceptable salt thereof is formulated (and administered) as a suspension of micro- or nanoparticles wherein the suspension comprises the following components in the following amounts:
(a) Rilpivirine (300 mg);
(b) Poloxamer 338 (50 mg); and
(c) Water for injection (ad 1 ml).

Alternatively, these components may be used in different amounts but with the same weight ratio between components and the total volume (made up by water for injection) scaled by the same value.

In a particular embodiment of the first aspect of the invention, the rilpivirine or pharmaceutically acceptable salt thereof is formulated (and administered) as a suspension of micro- or nanoparticles wherein the suspension comprises the following components in the following amounts:
a. Rilpivirine (300 mg);
b. Poloxamer 338 (50 mg);
c. Glucose monohydrate (19.25 mg);
d. Sodium dihydrogen phosphate (2.00 mg);
e. Citric acid monohydrate (1.00 mg);
f. Sodium Hydroxide (0.866 mg); and
g. Water for injection (ad 1 ml).

Alternatively, these components may be used in different amounts but with the same weight ratio between components and the total volume (made up by water for injection) scaled by the same value.

In an embodiment of the first aspect of the invention, the suspension of rilpivirine or a pharmaceutically acceptable salt thereof as described herein is administered by a manual injection process.

In an embodiment of the ninth aspect of the invention, the amount of the rilpivirine or pharmaceutically acceptable salt thereof in the suspension or the pharmaceutical composition of the invention is from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg, or from about 2700 mg to about 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment, the suspension of the ninth aspect of the invention is formulated for administration by subcutaneous or intramuscular injection. In a preferred embodiment of the ninth aspect of the invention, the suspension of the invention is formulated for administration by subcutaneous injection.

In a particular embodiment, the rilpivirine or a pharmaceutically acceptable salt thereof of the ninth aspect of the invention is formulated in a formulation comprising the following components:
rilpivirine or a pharmaceutically acceptable salt thereof, in particular rilpivirine in suspension as defined herein;
a surface modifier as defined herein, in particular poloxamer 338;
an isotonizing agent, in particular glucose monohydrate;
a buffer, in particular sodium dihydrogen phosphate;
a chelating agent, in particular citric acid monohydrate;
a pH adjusting agent, in particular sodium hydroxide; and
water, in particular water for injection.

In a particular embodiment, the rilpivirine or a pharmaceutically acceptable salt thereof of the ninth aspect of the invention is formulated in a formulation comprising the following components:
rilpivirine or a pharmaceutically acceptable salt thereof, in particular rilpivirine in suspension as defined herein;
poloxamer 338;
glucose monohydrate;
sodium dihydrogen phosphate;
citric acid monohydrate;
sodium hydroxide; and
water, in particular water for injection.

In a particular embodiment of the ninth aspect of the invention, the rilpivirine or pharmaceutically acceptable salt thereof is formulated as a suspension of micro- or nanoparticles wherein the suspension comprises the following components in the following amounts:
(a) Rilpivirine (300 mg);
(b) Poloxamer 338 (50 mg); and
(c) Water for injection (ad 1 ml).

Alternatively, these components may be used in different amounts but with the same weight ratio between components and the total volume (made up by water for injection) scaled by the same value.

In a particular embodiment, the rilpivirine or a pharmaceutically acceptable salt thereof of the ninth aspect of the invention is formulated in a formulation comprising the following components in the following amounts:
(a) Rilpivirine (300 mg) in the form of micro- or nanoparticles in suspension as defined herein;
(b) Poloxamer 338 (50 mg);
(c) Glucose monohydrate (19.25 mg);
(d) Sodium dihydrogen phosphate (2.00 mg);
(e) Citric acid monohydrate (1.00 mg);
(f) Sodium Hydroxide (0.866 mg); and
(g) Water for injection (ad 1 ml).

Alternatively, these components may be used in different amounts but with the same weight ratio between components and the total volume (made up by water for injection) scaled by the same value.

For the avoidance of doubt, each of the embodiments described in this section in relation to the first aspect of the invention applies equally to, i.e. is also disclosed in combination with, aspects two to eight of the invention. Further, each of the embodiments described in this section in relation to the ninth aspect of the invention applies equally to, i.e. is also disclosed in combination with aspects ten to thirteen of the invention.

Hyaluronidase

Hyaluronidase is an enzyme that degrades hyaluronic acid (HA) and lowers the viscosity of hyaluronan in the extracellular matrix. Because of this property, it can be used to increase dispersion and absorption of injected active pharmaceutical ingredients. Enzymatic activity of hyaluronidase, including rHuPH20, can be defined by units per mL (U/mL) or by total enzyme activity in a particular formulation (U).

It is generally known that the delivery of hyaluronidases (E.C. 3.2.1.35/36) into the tissue improves the penetration of drugs. Administration of hyaluronidase thus represents a method of increasing the dispersion and improving the absorption of drugs.

Administering high volumes of rilpivirine or a pharmaceutically acceptable salt thereof may result in bump formation at injection sites. Administration of a hyaluronidase with rilpivirine or a pharmaceutically acceptable salt thereof according to the first aspect of the invention may result in a reduction of such bump formation.

The term “hyaluronidase” as used herein means any enzyme that degrades hyaluronic acid and lowers the viscosity of hyaluronan in the extracellular matrix.

In a preferred embodiment of the first aspect of the invention, the hyaluronidase is recombinant hyaluronidase. In a particularly preferred embodiment of the first aspect of the invention, the hyaluronidase is recombinant human hyaluronidase, e.g. rHuPH20. In an embodiment of the first aspect of the invention, rHuPH20 is defined by the amino acid sequence available under CAS Registry No. 757971-58-7. Further information regarding rHuPH20 is provided in Int. Pat. Publ. No. WO2004/078140. In an embodiment of the first aspect of the invention, the amino acid sequence of rHuPH20 comprises SEQ ID NO: 1. In some embodiments of the first aspect of the invention, the hyaluronidase is a variant of rHuPH20 having an amino acid sequence of rHuPH20 that comprises SEQ ID NO: 2, namely residues 36-482 of wild type human hyaluronidase. In some embodiments of the first aspect of the invention, the hyaluronidase is a variant of rHuPH20 having an the amino acid sequence that comprises SEQ ID NO: 3. In some embodiments of the first aspect of the invention, the hyaluronidase is a variant of rHuPH20 having an amino acid sequence that comprises SEQ ID NO: 4. In some embodiments of the first aspect of the invention, the hyaluronidase is a variant of rHuPH20 having an the amino acid sequence that comprises SEQ ID NO: 5.

| | |
|---|---|
| SEQ ID NO: 1: | LNFRAPPVIPNVPFLWAWNAPSEFCLGKFDEPLDMSLFSFIGSPRIN |
| rHuPH20 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEP |
| SEQ ID NO: 2: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 1 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIFY |
| SEQ ID NO: 3: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 2 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIF |
| SEQ ID NO: 4: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 3 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQI |
| SEQ ID NO: 5: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 4 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQ |

In an embodiment of the first aspect of the invention, the hyaluronidase of the invention is formulated in a separate pharmaceutical composition. As discussed further herein (e.g. in the section titled “Use of rilpivirine or a pharmaceutically acceptable salt thereof and hyaluronidase in the first to eighth aspects of the invention and rilpivirine or a pharmaceutically acceptable salt thereof in the ninth to thirteenth aspects of the invention”), the separate pharmaceutical composition may be administered sequentially with a pharmaceutical composition comprising the rilpivirine or pharmaceutically acceptable salt thereof, or the separate pharmaceutical composition may be admixed extemporaneously with a pharmaceutical composition comprising the rilpivirine or pharmaceutically acceptable salt thereof prior to administration of the resulting admixed pharmaceutical composition.

In another embodiment, the hyaluronidase of the first aspect of the invention is formulated in the same pharmaceutical composition as the rilpivirine or pharmaceutically acceptable salt thereof, i.e. the hyaluronidase is formulated as a combined pharmaceutical composition (with the rilpivirine or pharmaceutically acceptable salt thereof).

In an embodiment of the first aspect of the invention, the hyaluronidase is in the form of a solution, preferably wherein the concentration of the hyaluronidase in the solution is from about 50 to about 20,000 U/mL, preferably about 50 to about 10,000 U/mL, from about 50 to about 5000 U/mL, from about 500 to about 2000 U/mL. In an embodiment of the first aspect of the invention, the hyaluronidase is in the form of a solution, preferably wherein the concentration of the hyaluronidase in the solution is about 500 U/mL. In an embodiment of the first aspect of the invention, the hyaluronidase is in the form of a solution, preferably wherein the concentration of the hyaluronidase in the solution is about 750 U/mL. In an embodiment of the first aspect of the invention, the hyaluronidase is in the form of a solution, preferably wherein the concentration of the hyaluronidase in the solution is about 1000 U/mL. In an embodiment of the first aspect of the invention, the hyaluronidase is in the form of a solution, preferably wherein the concentration of the hyaluronidase in the solution is about 1250 U/mL. In an embodiment of the first aspect of the invention, the hyaluronidase is in the form of a solution, preferably wherein the concentration of the hyaluronidase in the solution is about 1500 U/mL. In an embodiment of the first aspect of the invention, the hyaluronidase is in the form of a solution, preferably wherein the concentration of the hyaluronidase in the solution is about 1750 U/mL. In an embodiment of the first aspect of the invention, the hyaluronidase is in the form of a solution, preferably wherein the concentration of the hyaluronidase in the solution is about 2000 U/mL.

In some embodiments of the first aspect of the invention, the hyaluronidase containing composition comprises hyaluronidase at a dose of about 1,000 U, 2,000 U, 3,000 U, 4,000 U, about 5,000 U, about 6,000 U, about 7,000 U, about 8,000 U, about 9,000 U, about U, about 11,000 U, about 12,000 U, about 13,000 U, about 14,000 U, about 15,000 U, about 16,000 U, about 17,000 U, about 18,000 U, about 19,000 U, about 20,000 U, about 21,000 U, about 22,000 U, about 23,000 U, about 24,000 U, about 25,000 U, about 26,000 U, about 27,000 U, about 30,000 U, about 31,000 U, about 32,000 U, about 33,000 U, about 34,000 U, about 35,000 U, about 36,000 U, about 37,000 U, about 38,000 U, about 39,000 U, about 40,000 U, or any value in between. In some embodiments of the first aspect of the invention, where the hyaluronidase is administered sequentially with a pharmaceutical composition comprising the rilpivirine or pharmaceutically acceptable salt thereof, the hyaluronidase containing composition comprises hyaluronidase at a dose of about 1,000 U, 2,000 U, 3,000 U, 4,000 U, about 5,000 U, about 6,000 U, about 7,000 U, about 8,000 U, about 9,000 U, about 10,000 U, or any value in between. In a preferred embodiment of the first aspect of the invention the hyaluronidase containing composition comprises hyaluronidase at a dose of about 2,000 U. In some embodiments of the first aspect of the invention, where the hyaluronidase is admixed extemporaneously with a pharmaceutical composition comprising the rilpivirine or pharmaceutically acceptable salt thereof prior to administration of the resulting admixed pharmaceutical composition, the admixed composition comprises hyaluronidase at a dose of about 11,000 U, about 12,000 U, about 13,000 U, about 14,000 U, about 15,000 U, about 16,000 U, about 17,000 U, about 18,000 U, about 19,000 U, about 20,000 U, about 21,000 U, about 22,000 U, about 23,000 U, about 24,000 U, about 25,000 U, about 26,000 U, about 27,000 U, about 30,000 U, about 31,000 U, about 32,000 U, about 33,000 U, about 34,000 U, about 35,000 U, about 36,000 U, about 37,000 U, about 38,000 U, about 39,000 U, about 40,000 U, or any value in between. In a preferred embodiment of the first aspect of the invention, the admixed composition comprises hyaluronidase at a dose of about 18,000 U or 30,000 U.

In a particular embodiment of the first aspect of the invention, the hyaluronidase is formulated as a solution in a separate pharmaceutical composition, i.e. as a solution without the rilpivirine or a pharmaceutically acceptable salt thereof, and the separate pharmaceutical composition comprises the following components:
from about 50 U/mL to about 10,000 U/mL rHuPH20;
from about 5 mM to about 50 mM histidine;
from about 50 mM to about 400 mM sorbitol;
from about 0.1 mg/mL to about 2.5 mg/mL methionine; and
from about 0.01% (w/v) to about 0.1% (w/v) polysorbate 20 buffer.

For the avoidance of doubt, each of the embodiments described in this section in relation to the first aspect of the invention applies equally to, i.e. is also disclosed in combination with aspects two to eight of the invention.

Use of Rilpivirine or a Pharmaceutically Acceptable Salt Thereof and Hyaluronidase in the First to Eighth Aspects of the Invention and Rilpivirine or a Pharmaceutically Acceptable Salt Thereof in the Ninth to Thirteenth Aspects of the Invention

In a first aspect of the invention there is provided a method for the treatment or prevention of HIV infection in a subject in need thereof, the method comprising administering to the subject a therapeutically effective amount of rilpivirine or a pharmaceutically acceptable salt thereof in the form of micro- or nanoparticles in suspension by intramuscular injection or subcutaneous injection, wherein the rilpivirine or pharmaceutically acceptable salt thereof is administered in combination with a hyaluronidase that is administered by intramuscular injection or subcutaneous injection, and wherein the rilpivirine or pharmaceutically acceptable salt thereof and the hyaluronidase are administered intermittently at a time interval of about three months to about two years.

Thus, the method for treatment or prevention of the first aspect of the invention described herein involves administering rilpivirine or a pharmaceutically acceptable salt thereof and a hyaluronidase multiple times, and the time interval between an administration of the rilpivirine or pharmaceutically acceptable salt thereof and the hyaluronidase and a subsequent administration of the rilpivirine or pharmaceutically acceptable salt thereof and the hyaluronidase is about three months to about two years, i.e. the rilpivirine or pharmaceutically acceptable salt thereof and hyaluronidase according to the first aspect of the invention is administered to a subject as described herein, and then after a period of from three months to two years the rilpivirine or pharmaceutically acceptable salt thereof and hyaluronidase according to the invention is administered again to the subject as defined herein.

In an eleventh aspect of the invention there is provided rilpivirine or a pharmaceutically acceptable salt thereof according to the ninth aspect of the invention, i.e. in the form of micro- or nanoparticles in suspension, wherein the micro- or nanoparticles have a Dᵥ90 of from about 1 μm to about 10 μm, for use in the treatment or prevention of HIV infection in a subject.

The terms “is administered” and “are administered” as used herein in relation to the methods for treatment or prevention and uses described herein may encompass the terms “is to be administered” and “are to be administered”, respectively.

In a preferred embodiment of the first or eleventh aspect of the invention, the subject is a human.

The rilpivirine or pharmaceutically acceptable salt thereof and the hyaluronidase of the first aspect of the invention may be administered simultaneously or sequentially. In an embodiment of the first aspect of the invention, the rilpivirine or pharmaceutically acceptable salt thereof and the hyaluronidase are administered sequentially, i.e. one after the other, preferably within 24 hours of each other, preferably within 1 hour of each other, preferably within 30 minutes of each other, preferably within 10 minutes of each other, more preferably within 5 minutes of each other. Preferably, the hyaluronidase is administered before administration of the rilpivirine or pharmaceutically acceptable salt thereof. In another embodiment of the first aspect of the invention, the rilpivirine or pharmaceutically acceptable salt thereof and the hyaluronidase are administered simultaneously.

When the rilpivirine or pharmaceutically acceptable salt thereof and the hyaluronidase of the first aspect of the invention are administered sequentially, they are formulated in separate pharmaceutical compositions. These separate pharmaceutical compositions are described further in the sections titled “Rilpivirine” and “Hyaluronidase” herein.

When the rilpivirine or pharmaceutically acceptable salt thereof and the hyaluronidase of the first aspect of the invention are administered sequentially, they are both administered by the same method, i.e. subcutaneous or intramuscular injection. Further, they are both administered at the same site. By same site it is meant that the injection sites are within cm of each other, within 12 cm of each other, or within 8 cm of each other. Preferably the injection sites are within 10 cm of each other, more preferably within 5 cm of each other, even more preferably within 1 cm of each other. This allows the hyaluronidase to exert its effect in increasing the tolerability of the injection of rilpivirine or pharmaceutically acceptable salt thereof.

When the rilpivirine or pharmaceutically acceptable salt thereof and hyaluronidase of the first aspect of the invention are administered simultaneously, they may both be administered at the same site, i.e. simultaneously via the same syringe/needle. When the rilpivirine or pharmaceutically acceptable salt thereof and hyaluronidase of the first aspect of the invention are administered simultaneously, the rilpivirine or pharmaceutically acceptable salt thereof and hyaluronidase may be provided in combined pharmaceutical composition, i.e. a pharmaceutical composition comprising both the rilpivirine or pharmaceutically acceptable salt thereof and the hyaluronidase. This combined pharmaceutical composition is described further in the sections titled “Rilpivirine” and “Hyaluronidase” herein. When the rilpivirine or pharmaceutically acceptable salt thereof and hyaluronidase of the first aspect of the invention are administered simultaneously, the rilpivirine or pharmaceutically acceptable salt thereof and hyaluronidase may also be provided as separate pharmaceutical compositions which are admixed (i.e. to provide an admixed pharmaceutical formulation extemporaneously prior to administration).

The combined pharmaceutical composition of the first aspect of the invention is surprisingly stable on storage, i.e. the hyaluronidase is active even after being combined with rilpivirine or a pharmaceutically acceptable salt thereof, extemporaneously prior to administration, e.g. for at least 4 hours at room temperature, or for 24 hours or longer, in particular when stored at 2-8° C.

In an embodiment, the rilpivirine or a pharmaceutically acceptable salt thereof and the hyaluronidase of the first aspect of the invention are administered at the same injection site sequentially, through the same needle that has not been removed from the injection site, e.g. the skin.

The rilpivirine or pharmaceutically acceptable salt thereof and hyaluronidase of the first aspect of the invention are administered such that the time interval between administrations (i.e. the dosing interval) is about three months to about two years. That is, the rilpivirine or pharmaceutically acceptable salt thereof is administered (e.g. simultaneously or sequentially) with the hyaluronidase and then following a time interval of about three months to about one year the rilpivirine or pharmaceutically acceptable salt thereof is administered (e.g. simultaneously or sequentially) with the hyaluronidase again.

It has been found that the extended, sustained or prolonged release of rilpivirine when administered in the form of micro- or nanoparticles in suspension by intramuscular or subcutaneous injection can be maintained when administering rilpivirine or pharmaceutically acceptable salt thereof with a hyaluronidase of the first aspect of the invention as defined herein. This surprising effect is discussed in detail in Examples 1 and 2.

In an embodiment, the treatments or preventions of the eleventh aspect of the invention involve administering rilpivirine or a pharmaceutically acceptable salt thereof multiple times, i.e. intermittently, and the time interval between an administration of the rilpivirine or pharmaceutically acceptable salt thereof and a subsequent administration of the rilpivirine or pharmaceutically acceptable salt thereof (i.e. the dosing interval) is about three months to about two years, i.e. the rilpivirine or pharmaceutically acceptable salt thereof according to the eleventh aspect of the invention is administered to a subject as described herein, and then after a period of from about three months to about two years the rilpivirine or pharmaceutically acceptable salt thereof according to the eleventh aspect of the invention is administered again to the subject as defined herein.

In an embodiment of the first and eleventh aspects of the invention, the time interval described herein is about 1.5 years. In an embodiment of the first and eleventh aspects of the invention, the time interval described herein is about two years. In a preferred embodiment of the first and eleventh aspects of the invention, the time interval described herein is about three months to about 1.5 years. In another preferred embodiment of the first and eleventh aspects of the invention, the time interval described herein is about three months to about one year. In another preferred embodiment of the first and eleventh aspects of the invention, the time interval described herein is about three months to about six months. In another preferred embodiment of the first and eleventh aspects of the invention, the time interval described herein is about six months to about 1 year. In another preferred embodiment of the first and eleventh aspects of the invention, the time interval described herein is about three months. In another preferred embodiment of the first and eleventh aspects of the invention, the time interval described herein is about six months. In another preferred embodiment of the first and eleventh aspects of the invention, the time interval described herein is about 1 year.

The rilpivirine or pharmaceutically acceptable salt thereof and the hyaluronidase of the first aspect of the invention are administered by subcutaneous injection or intramuscular injection. Preferably, the rilpivirine and the hyaluronidase of the first aspect of the invention are administered by subcutaneous injection (either via the same combined pharmaceutical composition or via separate pharmaceutical compositions).

In an embodiment of the eleventh aspect of the invention, the rilpivirine or pharmaceutically acceptable salt thereof is administered by subcutaneous injection or intramuscular injection. Preferably, the rilpivirine or pharmaceutically acceptable salt thereof is administered by subcutaneous injection.

In an embodiment of the eleventh aspect of the invention, the rilpivirine or a pharmaceutically acceptable salt thereof is administered by a manual injection process.

The rilpivirine or pharmaceutically acceptable salt thereof and the hyaluronidase of the first aspect of the invention and the rilpivirine or pharmaceutically acceptable salt thereof of the eleventh aspect of the invention are used in a method for the treatment or prevention of HIV infection in a subject, i.e. the rilpivirine or pharmaceutically acceptable salt thereof and the hyaluronidase of the first aspect of the invention as defined herein and the rilpivirine or pharmaceutically acceptable salt thereof of the eleventh aspect of the invention as defined herein are for use in the treatment or prevention of HIV infection. The rilpivirine or pharmaceutically acceptable salt thereof is administered in a therapeutically effective amount. By “therapeutically effective amount” it is meant an amount sufficient to provide a therapeutic effect.

In a particular embodiment, the rilpivirine or a pharmaceutically acceptable salt thereof used in the first aspect of the invention is rilpivirine, and the rilpivirine and the hyaluronidase are used in a method for the treatment of HIV infection in a subject in need thereof as described herein, wherein the suspension comprises a pharmaceutically acceptable aqueous carrier in which the rilpivirine is suspended in the form of micro- or nanoparticles and wherein the rilpivirine and the hyaluronidase are administered by subcutaneous injection, preferably wherein the average effective particle size of the micro- or nanoparticles is from about 100 nm to about 300 nm, and preferably wherein a surface modifier, e.g. poloxamer 338, is adsorbed to the surface of the micro- or nanoparticles.

In a particular embodiment, the rilpivirine or a pharmaceutically acceptable salt thereof used in the first aspect of the invention is rilpivirine, and the rilpivirine and the hyaluronidase are used in a method for the treatment of HIV infection in a subject in need thereof as described herein, wherein the suspension comprises a pharmaceutically acceptable aqueous carrier in which the rilpivirine is suspended in the form of micro- or nanoparticles and wherein the rilpivirine and the hyaluronidase are administered by subcutaneous injection, preferably wherein the micro- or nanoparticles have a Dᵥ50 ranging of from about 0.2 μm to about 3 μm or having a Dᵥ50 as described herein, and preferably wherein a surface modifier, e.g. poloxamer 338, is adsorbed to the surface of the micro- or nanoparticles.

In a particular embodiment, the rilpivirine or a pharmaceutically acceptable salt thereof used in the first aspect of the invention is rilpivirine, and the rilpivirine and the hyaluronidase are used in a method for the treatment of HIV infection in a subject in need thereof as described herein, wherein the suspension comprises a pharmaceutically acceptable aqueous carrier in which the rilpivirine is suspended in the form of micro- or nanoparticles and wherein the rilpivirine and the hyaluronidase are administered by subcutaneous injection, preferably wherein the micro- or nanoparticles have a Dᵥ90 ranging of from about 1 μm to about 10 μm or having a Dᵥ90 as described herein, and preferably wherein a surface modifier, e.g. poloxamer 338, is adsorbed to the surface of the micro- or nanoparticles.

In a particular embodiment, the rilpivirine or a pharmaceutically acceptable salt thereof used in the eleventh aspect of the invention is rilpivirine, and the rilpivirine is used in a method for the treatment of HIV infection in a subject in need thereof as described herein, wherein the suspension comprises a pharmaceutically acceptable aqueous carrier in which the rilpivirine is suspended in the form of micro- or nanoparticles and wherein the rilpivirine is administered by subcutaneous injection, preferably wherein the micro- or nanoparticles have a Dᵥ50 ranging of from about 0.2 μm to about 3 μm in combination with a Dᵥ90 ranging of from about 1 μm to about 10 μm or having a combination of Dᵥ50 and Dᵥ90 as described herein, and preferably wherein a surface modifier, e.g. poloxamer 338, is adsorbed to the surface of the micro- or nanoparticles.

In a particular embodiment, the rilpivirine or a pharmaceutically acceptable salt thereof in the eleventh aspect of the invention is rilpivirine, and the rilpivirine is used for the treatment of HIV infection in a subject in need thereof as described herein, wherein the suspension comprises a pharmaceutically acceptable aqueous carrier in which the rilpivirine is suspended in the form of micro- or nanoparticles having a Dᵥ90 of from about 1 μm to about 7 μm, and wherein the rilpivirine is administered by subcutaneous injection, preferably wherein a surface modifier, e.g. poloxamer 338, is adsorbed to the surface of the micro- or nanoparticles.

In an embodiment, the rilpivirine or pharmaceutically acceptable salt thereof and the hyaluronidase of the first aspect of the invention and the rilpivirine or a pharmaceutically acceptable salt thereof of the eleventh aspect of the invention are used in a method for the treatment or prevention of HIV type 1 (HIV-1) infection in a subject, i.e. an embodiment described herein relates to the use of rilpivirine or pharmaceutically acceptable salt thereof and a hyaluronidase of the first aspect of the invention and use of rilpivirine or a pharmaceutically acceptable salt thereof of the eleventh aspect of the invention as defined herein for treating or preventing HIV type 1 (HIV-1) infection in a subject.

In an embodiment of the eleventh aspect of the invention, each administration comprises up to about 600 mL of the suspension described herein, i.e. the volume of the suspension comprising the rilpivirine or a pharmaceutically acceptable salt thereof may have a volume of up to 600 mL. In an embodiment of the eleventh aspect of the invention, each administration comprises from about 5 mL to about 600 mL of the suspension. In another embodiment of the eleventh aspect of the invention, each administration comprises from about 5 mL to about 300 mL of the suspension. In another embodiment of the eleventh aspect of the invention, each administration comprises from about 5 mL to about 150 mL of the suspension. In another embodiment of the eleventh aspect of the invention, each administration comprises from about 5 mL to about 25 mL of the suspension. In another embodiment of the eleventh aspect of the invention, each administration comprises from about 6 mL to about 20 mL of the suspension. In another embodiment of the eleventh aspect of the invention, each administration comprises from about 6 mL to about 18 mL of the suspension. In another embodiment of the eleventh aspect of the invention, each administration comprises from about 6 mL to about 15 mL of the suspension. In another embodiment of the eleventh aspect of the invention, each administration comprises from about 6 mL to about 12 mL of the suspension. In another embodiment of the eleventh aspect of the invention, each administration comprises from about 9 mL to about 18 mL of the suspension. In another embodiment of the eleventh aspect of the invention, each administration comprises from about 9 mL to about 15 mL of the suspension. In another embodiment of the eleventh aspect of the invention, each administration comprises from about 9 mL to about 12 mL of the suspension. In another embodiment of the eleventh aspect of the invention, each administration comprises about 6 mL of the suspension. In another embodiment of the eleventh aspect of the invention, each administration comprises about 9 mL of the suspension. In another embodiment of the eleventh aspect of the invention, each administration comprises about 12 mL of the suspension. In another embodiment of the eleventh aspect of the invention, each administration comprises about mL of the suspension. In another embodiment of the eleventh aspect of the invention, each administration comprises about 18 mL of the suspension. In an embodiment of the eleventh aspect of the invention, the rilpivirine suspension contains 300 mg rilpivirine/mL.

In an embodiment of the eleventh aspect of the invention, for the treatment of HIV infection, the dose to be administered may be calculated on a basis of about 300 mg to about 1200 mg/month, or about 450 mg to about 1200 mg/month, or about 450 mg to about 900 mg/month, or about 450 mg to about 750 mg/month, or about 600 mg to about 900 mg/month, or 450 mg/month, or 600 mg/month, or 750 mg/month, or 900 mg/month. Doses for other dosing regimens can readily be calculated by multiplying the monthly dose with the number of months between each administration. For example, in case of a dose of 450 mg/month, and in case of a time interval of 6 months between each administration, the dose to be administered in each administration is 2700 mg. Or for example, in case of a dose of 750 mg/month, and in case of a time interval of 6 months between each administration, the dose to be administered in each administration is 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment of the eleventh aspect of the invention, for the treatment of HIV infection, the dose to be administered may be calculated on a basis of about 300 mg to about 1200 mg/4 weeks (28 days), or about 450 mg to about 1200 mg/4 weeks (28 days), or about 450 mg to about 900 mg/4 weeks (28 days), or about 450 mg to about 750 mg/4 weeks (28 days), or about 600 mg to about 900 mg/4 weeks (28 days), or 450 mg/4 weeks (28 days), or 600 mg/4 weeks (28 days), or 750 mg/4 weeks (28 days), or 900 mg/4 weeks (28 days). Doses for other dosing regimens can readily be calculated by multiplying the week or day dose with the number of weeks between each administration. For example, in case of a dose of 450 mg/4 weeks (28 days), and in case of a time interval of 24 weeks between each administration, the dose to be administered in each administration is 2700 mg. Or for example, in case of a dose of 750 mg/4 weeks (28 days), and in case of a time interval of 24 weeks between each administration, the dose to be administered in each administration is 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment of the eleventh aspect of the invention, for the treatment of HIV infection, each administration of rilpivirine or a pharmaceutically acceptable salt thereof may comprise from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg, or from about 2700 mg to about 4500 mg of the rilpivirine or pharmaceutically acceptable salt thereof.

In the instance of prevention of HIV infection, each administration of rilpivirine or pharmaceutically acceptable salt thereof according to the eleventh aspect of the invention may comprise the same dosing as for therapeutic applications as described above.

In an embodiment of the eleventh aspect of the invention, the rilpivirine or pharmaceutically acceptable salt thereof is used in an amount such that the blood plasma concentration of rilpivirine in the subject is kept at a level above about 12 ng/ml, preferably ranging from about 12 ng/ml to about 100 ng/ml, more preferably about 12 ng/ml to about ng/ml for at least three months after administration, or at least 6 months after administration, or at least 9 months after administration, or at least 1 year after administration, or at least 2 years after each administration. In a preferred embodiment of the eleventh aspect of the invention, the rilpivirine or pharmaceutically acceptable salt thereof is used in an amount such that the blood plasma concentration of rilpivirine in the subject is kept at a level of from 12 ng/ml to 100 ng/ml for at least 6 months.

As used herein the term “treatment of HIV infection” relates to the treatment of a subject infected with HIV. The term “treatment of HIV infection” also relates to the treatment of diseases associated with HIV infection, for example AIDS, or other conditions associated with HIV infection including thrombocytopaenia, Kaposi's sarcoma and infection of the central nervous system characterized by progressive demyelination, resulting in dementia and symptoms such as, progressive dysarthria, ataxia and disorientation, and further conditions where HIV infection has also been associated with, such as peripheral neuropathy, progressive generalized lymphadenopathy (PGL), and AIDS-related complex (ARC).

As used herein the term “prevention of HIV infection” relates to the prevention or avoidance of a subject (who is not infected with HIV) becoming infected with HIV. The source of infection can be various, a material containing HIV, in particular a body fluid that contains HIV such as blood or semen, or another subject who is infected with HIV. Prevention of HIV infection relates to the prevention of the transmission of the virus from the material containing HIV or from the HIV infected individual to an uninfected person, or relates to the prevention of the virus from entering the body of an uninfected person. Transmission of the HIV virus can be by any known cause of HIV transfer such as by sexual transmission or by contact with blood of an infected subject, e.g. medical staff providing care to infected subjects. Transfer of HIV can also occur by contact with HIV infected blood, e.g. when handling blood samples or with blood transfusion. It can also be by contact with infected cells, e.g. when carrying out laboratory experiments with HIV infected cells.

The term “treatment of HIV infection” refers to a treatment by which the viral load of HIV (represented as the number of copies of viral RNA in a specified volume of serum) is reduced. The more effective the treatment, the lower the viral load. Preferably the viral load should be reduced to as low levels as possible, e.g. below about 200 copies/ml, in particular below about 100 copies/ml, more in particular below 50 copies/ml, if possible below the detection limit of the virus. Reductions of viral load of one, two or even three orders of magnitude (e.g. a reduction in the order of about 10 to about 10², or more, such as about 10³) are an indication of the effectiveness of the treatment. Another parameter to measure effectiveness of HIV treatment is the CD4 count, which in normal adults ranges from 500 to 1500 cells per μl. Lowered CD4 counts are an indication of HIV infection and once below about 200 cells per μl, AIDS may develop. An increase of CD4 count, e.g. with about 50, 100, 200 or more cells per μl, is also an indication of the effectiveness of anti-HIV treatment. The CD4 count in particular should be increased to a level above about 200 cells per μl, or above about 350 cells per μl. Viral load or CD4 count, or both, can be used to diagnose the degree of HIV infection.

The term “treatment of HIV infection” and similar terms refer to that treatment that lowers the viral load, or increases CD4 count, or both, as described above. The term “prevention of HIV infection” and similar terms refer to that situation where there is a decrease in the relative number of newly infected subjects in a population in contact with a source of HIV infection such as a material containing HIV, or a HIV infected subject. Effective prevention can be measured, for example, by measuring in a mixed population of HIV infected and non-infected individuals, if there is a decrease of the relative number of newly infected individuals, when comparing non-infected individuals treated with a pharmaceutical composition of the invention, and non-treated non-infected individuals. This decrease can be measured by statistical analysis of the numbers of infected and non-infected individuals in a given population over time.

In a second aspect there is provided rilpivirine or a pharmaceutically acceptable salt thereof and a hyaluronidase for use in the treatment or prevention of HIV infection in a subject, wherein the rilpivirine or pharmaceutically acceptable salt thereof is in the form of micro- or nanoparticles in suspension, wherein the rilpivirine or pharmaceutically acceptable salt thereof and hyaluronidase are administered to the subject by intramuscular injection or subcutaneous injection, and wherein the rilpivirine or pharmaceutically acceptable salt thereof and hyaluronidase are administered intermittently at a time interval of about three months to about two years.

It will be understood that all of the embodiments described herein in relation to the first aspect, e.g. the embodiments relating to the rilpivirine in the first aspect of the invention, hyaluronidase in the invention, and the uses of the rilpivirine and hyaluronidase in the first aspect of the invention, apply equivalently, i.e. are also disclosed herein in relation to, this second aspect of the invention.

In a third aspect there is provided products containing rilpivirine or a pharmaceutically acceptable salt thereof and a hyaluronidase as a combined preparation for simultaneous or sequential use in the treatment or prevention of HIV infection by intramuscular injection or subcutaneous injection, wherein the rilpivirine or pharmaceutically acceptable salt thereof is in the form of micro- or nanoparticles in suspension, and wherein the rilpivirine or a pharmaceutically acceptable salt thereof and the hyaluronidase are administered intermittently at a time interval of about three months to about two years.

It will be understood that all of the embodiments described herein in relation to the first aspect, e.g. the embodiments relating to the rilpivirine in the first aspect of the invention, hyaluronidase in the invention, and the uses of the rilpivirine and hyaluronidase in the first aspect of the invention, apply equivalently, i.e. are also disclosed herein in relation to, this third aspect of the invention.

In a fourth aspect there is provided a kit of parts comprising rilpivirine or a pharmaceutically acceptable salt thereof and a hyaluronidase for simultaneous or sequential use in the treatment or prevention of HIV infection by intramuscular injection or subcutaneous injection, wherein the rilpivirine or a pharmaceutically acceptable salt thereof is in the form of micro- or nanoparticles in suspension, and wherein the rilpivirine or a pharmaceutically acceptable salt thereof and a hyaluronidase are administered intermittently at a time interval of about three months to about two years.

It will be understood that all of the embodiments described herein in relation to the first aspect, e.g. the embodiments relating to the rilpivirine in the first aspect of the invention, hyaluronidase in the invention, and the uses of the rilpivirine and hyaluronidase in the first aspect of the invention, apply equivalently, i.e. are also disclosed herein in relation to, this fourth aspect of the invention.

In a fifth aspect there is provided rilpivirine or a pharmaceutically acceptable salt thereof in the form of micro- or nanoparticles in suspension for use in the treatment or prevention of HIV infection by intramuscular injection or subcutaneous injection, wherein the rilpivirine or pharmaceutically acceptable salt thereof is administered in combination with a hyaluronidase that is administered by intramuscular injection or subcutaneous injection, and wherein the rilpivirine or pharmaceutically acceptable salt thereof and the hyaluronidase are administered intermittently at a time interval of about three months to about two years.

It will be understood that all of the embodiments described herein in relation to the first aspect, e.g. the embodiments relating to the rilpivirine in the first aspect of the invention, hyaluronidase in the invention, and the uses of the rilpivirine and hyaluronidase in the first aspect of the invention, apply equivalently, i.e. are also disclosed herein in relation to, this fifth aspect of the invention.

In a sixth aspect there is provided use of rilpivirine or a pharmaceutically acceptable salt thereof for the manufacture of a medicament for treating or preventing HIV infection in a subject, wherein the rilpivirine or pharmaceutically acceptable salt thereof is in the form of micro- or nanoparticles in suspension and is administered in combination with a hyaluronidase, wherein the rilpivirine or pharmaceutically acceptable salt thereof and the hyaluronidase are administered to the subject by intramuscular injection or subcutaneous injection, and wherein the rilpivirine or pharmaceutically acceptable salt thereof and the hyaluronidase are administered intermittently at a time interval of about three months to about two years.

It will be understood that all of the embodiments described herein in relation to the first aspect, e.g. the embodiments relating to the rilpivirine in the first aspect of the invention, hyaluronidase in the first aspect of the invention, and the uses of the rilpivirine and hyaluronidase in the invention, apply equivalently, i.e. are also disclosed herein in relation to, this sixth aspect of the invention.

In a seventh aspect there is provided a combination comprising rilpivirine or a pharmaceutically acceptable salt thereof and a hyaluronidase, wherein the rilpivirine or a pharmaceutically acceptable salt thereof is in the form of micro- or nanoparticles in suspension.

It will be understood that all of the embodiments described herein in relation to the first aspect, e.g. the embodiments relating to the rilpivirine in the first aspect of the invention and the hyaluronidase in the first aspect of the invention apply equivalently, i.e. are also disclosed herein in relation to, this seventh aspect of the invention.

In some embodiments, there is provided the combination of the seventh aspect of the invention for use in the treatment or prevention of HIV infection, wherein the combination is administered intermittently by intramuscular injection or subcutaneous injection at a time interval of about three months to about two years.

In an eighth aspect there is provided a kit of parts comprising rilpivirine or a pharmaceutically acceptable salt thereof and a hyaluronidase, wherein the rilpivirine or a pharmaceutically acceptable salt thereof is in the form of micro- or nanoparticles in suspension.

It will be understood that all of the embodiments described herein in relation to the first aspect, e.g. the embodiments relating to the rilpivirine in the first aspect of the invention and the hyaluronidase in the first aspect of the invention apply equivalently, i.e. are also disclosed herein in relation to, this eighth aspect of the invention.

In a twelfth aspect there is provided a method for treating or preventing HIV infection in a subject, the method comprising administering rilpivirine or a pharmaceutically acceptable salt thereof according to the ninth aspect of the invention, i.e. in the form of micro- or nanoparticles in suspension, wherein the micro- or nanoparticles have a Dᵥ90 of from about 1 μm to about 10 μm, to the subject.

It will be understood that all of the embodiments described herein in relation to the eleventh aspect, e.g. the embodiments relating to the rilpivirine in the eleventh aspect of the invention, apply equivalently, i.e. are also disclosed herein in relation to, this twelfth aspect of the invention.

In a thirteenth aspect there is provided use of rilpivirine or a pharmaceutically acceptable salt thereof according to the ninth aspect of the invention, i.e. in the form of micro- or nanoparticles in suspension, wherein the micro- or nanoparticles have a Dᵥ90 of from about 1 μm to about 10 μm, for the manufacture of a medicament for treating or preventing HIV infection in a subject.

It will be understood that all of the embodiments described herein in relation to the eleventh aspect, e.g. the embodiments relating to the rilpivirine in the eleventh aspect of the invention, apply equivalently, i.e. are also disclosed herein in relation to, this thirteenth aspect of the invention.

In an embodiment of the first to eighth aspects of the invention, the method or use or combination or products or kit of parts as described herein are used in combination with one or more other active agents, in particular one or more other antiretroviral agents, in particular one or more other antiretroviral agents of another class, such as for example an antiretroviral of the INSTI class, such as for example cabotegravir. In an embodiment of the first to eighth aspects of the invention, said one or more other antiretroviral agents, e.g. cabotegravir, is administered as an intramuscular or subcutaneous injection, in particular as an injectable micro- or nanosuspension, at a time interval of about three months to about two years. In an embodiment of the first to eighth aspects of the invention, said one or more other antiretroviral agent, e.g. cabotegravir, is administered at the same intermittent time interval as the rilpivirine or a pharmaceutically acceptable salt thereof and the hyaluronidase of the first to eighth aspects of the invention as described herein, e.g. the rilpivirine or a pharmaceutically acceptable salt thereof, hyaluronidase and the other antiretroviral agent are administered intermittently at a time interval of about three months, or of about four months, or of about five months or of about six months or of about seven months or of about eight months or of about ten months or of about eleven months or of about one year or of about one year to about 2 years. In an embodiment of the first to eighth aspects of the invention the rilpivirine or a pharmaceutically acceptable salt thereof, the hyaluronidase and the one or more other antiretroviral agents, e.g. cabotegravir, are administered simultaneously or sequentially by intramuscular or subcutaneous injection, in particular subcutaneous injection. In an embodiment of the first to eighth aspects of the invention the rilpivirine or a pharmaceutically acceptable salt thereof, the hyaluronidase and the one or more other antiretroviral agents, e.g. cabotegravir, are administered simultaneously, in particular by subcutaneous injection. In an embodiment of the first to eighth aspects of the invention the rilpivirine or a pharmaceutically acceptable salt thereof, the hyaluronidase and the one or more other antiretroviral agents, e.g. cabotegravir, are administered sequentially, in particular by subcutaneous injection. In an embodiment of the first to eighth aspects of the invention, the hyaluronidase is administered first followed by the rilpivirine or a pharmaceutically acceptable salt thereof followed by a cabotegravir injection. In an embodiment of the first to eighth aspects of the invention, the hyaluronidase is administered first followed by a cabotegravir injection followed by the rilpivirine or a pharmaceutically acceptable salt thereof.

In an embodiment of the eleventh to thirteenth aspects of the invention, the treatments/preventions of the invention are used in combination with one or more other active agents, in particular one or more other antiretroviral agents, in particular one or more other antiretroviral agents of another class, such as for example an antiretroviral of the INSTI class, such as for example cabotegravir. In an embodiment of the eleventh to thirteenth aspects of the invention, said one or more other antiretroviral agents, e.g. cabotegravir, is administered as an intramuscular or subcutaneous injection, in particular as an injectable micro- or nanosuspension, at a time interval of about three months to about two years. In an embodiment of the eleventh to thirteenth aspects of the invention, said one or more other antiretroviral agent, e.g. cabotegravir, is administered at the same intermittent time interval as the rilpivirine or a pharmaceutically acceptable salt thereof as described herein, e.g. the rilpivirine or a pharmaceutically acceptable salt thereof and the other antiretroviral agent are administered intermittently at a time interval of about three months, or of about four months, or of about five months or of about six months or of about seven months or of about eight months or of about ten months or of about eleven months or of about one year or of about one year to about 2 years. In an embodiment of the eleventh to thirteenth aspects of the invention the rilpivirine or a pharmaceutically acceptable salt thereof and the one or more other antiretroviral agents, e.g. cabotegravir, are administered simultaneously or sequentially by intramuscular or subcutaneous injection, in particular subcutaneous injection. In an embodiment of the eleventh to thirteenth aspects of the invention the rilpivirine or a pharmaceutically acceptable salt thereof and the one or more other antiretroviral agents, e.g. cabotegravir, are administered simultaneously, in particular by subcutaneous injection. In an embodiment of the eleventh to thirteenth aspects of the invention the rilpivirine or a pharmaceutically acceptable salt thereof and the one or more other antiretroviral agents, e.g. cabotegravir, are administered sequentially, in particular by subcutaneous injection. In an embodiment of the eleventh to thirteenth aspects of the invention, the rilpivirine or a pharmaceutically acceptable salt thereof is administered first followed by a cabotegravir injection. In an embodiment of the eleventh to thirteenth aspects of the invention, the cabotegravir injection is administered first followed by the rilpivirine or a pharmaceutically acceptable salt thereof.

For the avoidance of doubt, the pharmaceutical composition according to the tenth aspect of the invention can also be used in the treatments or preventions according to the eleventh to thirteenth aspects of the invention.

### General Definitions

The term “comprising” encompasses “including” as well as “consisting”, e.g. a composition “comprising” X may consist exclusively of X or may include something additional, e.g. X+Y. The term “comprising” used herein also encompasses “consisting essentially of”, e.g. a composition “comprising” X may consist of X and any other components that do not materially affect the essential characteristics of the composition.

The term “about” in relation to a numerical value Y is optional and means, for example, Y±10%.

When a time interval is expressed as a specified number of months, it runs from a given numbered day of a given month to the same numbered day of the month that falls the specified number of months later. Where the same numbered day does not exist in the month that falls the specified number of months later, the time interval runs into the following month for the same number of days it would have run if the same numbered day would exist in the month that falls the specified number of months later.

When a time interval is expressed as a number of years, it runs from a given date of a given year to the same date in the year that falls the specified number of years later. Where the same date does not exist in the year that falls the specified number of years later, the time interval runs for the same number of days it would have run if the same numbered day would exist in the month that falls the specified number of months later. In other words, if the time interval starts on 29th February of a given year but ends in a year where there is no 29th February, the time period ends instead on 1st March in that year. The term “about” in relation to such a definition means that the time interval may end on a date that is ±10% of the time interval.

In an embodiment, the time interval may start up to 7 days before or after the start of the time interval and end up to 7 days before or after the end of the time interval.

All references cited herein are incorporated by reference in their entirety.

The invention will now be described with reference to the following examples. For the avoidance of doubt, these examples do not limit the scope of the invention. Modifications may be made whilst remaining within the scope and spirit of the invention.

### EXAMPLES

### Example 1—Administration of Rilpivirine with a Hyaluronidase

This example compares the plasma kinetics after administration of a suspension of rilpivirine with the plasma kinetics following sequential administration of first a hyaluronidase solution then a rilpivirine suspension.

### Preparation of Rilpivirine and Hyaluronidase Compositions

(a) Suspension of Rilpivirine

A 3.380 mL fill of 300 mg/mL suspension of rilpivirine (Dᵥ50=−200 nm) was prepared in 4R glass vials with the following excipients:
Poloxamer 338 (50 mg/ml)
Glucose monohydrate (19.25 mg/ml)
Sodium dihydrogen phosphate monohydrate (2.00 mg/ml)
Citric acid monohydrate (1.00 mg/ml)
Sodium hydroxide (0.866 mg/ml)
Water for injection (q.s ad 3 mL)

The suspension was prepared as follows:

A buffer solution was prepared by dissolving citric acid monohydrate, sodium dihydrogen phosphate monohydrate, sodium hydroxide and, glucose monohydrate in water for injection in a stainless steel vessel. Poloxamer 338 was added to the buffer solution and mixed until dissolved. A first fraction of the poloxamer 338 buffer solution was passed sequentially through a pre-filter and 2 serially-connected sterile filters into a sterilized stainless steel vessel. The sterile drug substance (micronized irradiated) was aseptically dispersed, via a charging isolator, into the sterile solution. The remaining fraction of poloxamer 338 buffer solution was passed sequentially through a pre-filter and 2 serially-connected sterile filters into the milling vessel to make up the suspension concentrate. During and after addition of the drug substance, the suspension concentrate was mixed to wet and disperse the drug substance.

Milling of the Suspension Concentrate

The suspension concentrate in the milling vessel was aseptically milled by circulating through a sterilized stainless-steel milling chamber, using sterilized zirconia beads as grinding media. During the milling process, the suspension circulated between the milling chamber and the milling vessel by means of a peristaltic pump until the target particle size was achieved.

Dilution of the Suspension Concentrate to Final Concentration

The suspension concentrate in the holding vessel was diluted with water for injection, which is sterile filtered through a pre-filter and 2 serially connected sterile filters into this vessel via the milling chamber and the 70 μm stainless steel filter. After final dilution, the vessel headspace is blanketed with nitrogen and the suspension was mixed until homogeneous.

Holding and Filling of the Final Suspension

While mixing, the suspension was aseptically transferred from the holding vessel to the time/pressure (t/p) dosing vessel, from which the suspension was filled into vials which were flushed with nitrogen, stoppered and capped with an aluminium seal with a flip-off button.

(b) Solution of Hyaluronidase (rHuPH20)

A solution of rHuPH20 was prepared by diluting rHuPH20 concentrate (1×10⁶) to 10,000 U/mL by addition of 10 mM histidine, 300 mM sorbitol, 1 mg/mL methionine, pH 5.6, 0.04% polysorbate 20 buffer.

The solution was sterile filtered and provided in 1 mL aliquot of 10,000 U/mL filled into 2R sterile glass vials.

Procedure

Six minipigs with body weights ranging from 20 to 25 kg at the start of the study were used. The minipigs were fasted overnight before dosing. Three minipigs were dosed subcutaneously in the loin with 0.19 mL of the hyaluronidase solution (10,000 U/mL) followed by 900 mg/3 mL of the rilpivirine nanosuspension at the same injection site (treatment group A). Three minipigs were dosed subcutaneously in the loin with the 900 mg/3 mL of the control rilpivirine suspension (treatment group B—control). The injection volume was 3 mL rilpivirine suspension in both treatment groups.

Method—Sequential Administration
1. Flip-off rHuPH20 solution vial flip cap and wipe with isopropyl alcohol wipe. Allow to dry. Attach an 18 G transfer needle to a 1 mL syringe.
2. Draw 0.35 mL into the syringe.
3. Prime the syringe and set liquid level to 0.25 mL in the syringe.
4. Remove transfer needle and attach syringe cap to 1 mL syringe.
5. Mix rilpivirine by horizontally shaking the container 30 times over approximately within approximately 10 s (a back and forth arm movement=2 times). Ensure well mixed/fully re-suspended.
6. Flip-off rilpivirine vial flip cap and wipe with isopropyl alcohol wipe. Allow to dry.
7. Attach 18 G transfer needle to 5 mL syringe.
8. Invert vial and draw>3.2 mL into 5 mL syringe mL (or as much as can be removed from vial). Injecting 1-2 mL of air will facilitate draw.
9. Detach needle and attach a syringe cap to 5 mL syringe
10. Invert syringe. Wait 5 minutes for bubbles to settle.
11. Attach a winged infusion set to the rHuPH20 1 mL syringe. Invert syringe and prime off air so that liquid forms at the tip of the needle. (0.19 mL of rHuPH20 should be in the line)
12. Insert the winged infusion set into the subcutaneous tissue of the target injection site by pinching the skin and inserting the needle at a 30-45 degree angle.
13. Release the pinch.
14. UNSCREW rHuPH20 syringe from infusion set maintaining the needle in the skin. Keep luer end (open end) pointed upwards so the liquid does not drain out of the infusion line while preparing the rilpivirine syringe. It is advisable to have this syringe prepared while the technician is inserting the rHuPH20 infusion line.
15. Remove the syringe cap from the 5 mL syringe with rilpivirine. Remove air and set the dose at 3.2 mL.
16. Attach rilpivirine filled syringe to the open end of the infusion set.
17. Inject over 1 minute at a constant rate until the syringe plunger bottoms out (this will leave approximately 0.19 mL of rilpivirine in infusion line)
18. Remove the winged infusion set and dispose.
19. Record any site leakage.

Photography of Injection Site
Injection site protrusions were assessed visually.

Blood Sampling
Blood samples of 2 mL were taken from the jugular vein from all minipigs at time intervals over the following 2160 hours. Blood samples were placed on EDTA. Within 1 hour of blood sampling, samples were centrifuged at 5° C. at about 1900×g for ±10 minutes to allow plasma separation. Plasma was immediately transferred into a second tube and stored in the freezer within 1 hour after the start of centrifugation. Plasma samples were analysed individually by means of a validated LC-MS/MS method.

Pharmacokinetic Data Analysis
The pharmacokinetic profile of the blood plasma samples was evaluated using non-compartmental pharmacokinetic analysis (using individual Cₚ vs time profiles). Mean plasma concentrations and PK parameters (Cₘₐₓ, Tₘₐₓ, t_{1/2} and AUC values) were measured and the results are provided in Table 1.

Results and Discussion

TABLE 1
Pharmacokinetic parameters

| | rHuPH20 + rilpivirine | | Rilpivirine | |
|---|---|---|---|---|
| | (treatment group A) | | (treatment group B) | |
| Parameter | according to the invention | | control | |
| N | 3 | | 3 | |
| Cₘₐₓ (ng/mL) | 52.2 ± 24.1 | | 28.5 ± 9.56 | |
| Tₘₐₓ^{a} (h) | 312 | (24-312) | 744 | (312-1248) |
| Tₗₐₛₜ^{a} (h) | 2160 | (2160-2160) | 2160 | (2160-2160) |

| | | |
|---|---|---|
| AUCₗₐₛₜ (ng*h/mL) | 24900 ± 7840 | 22330 ± 2930 |
| AUC_{∞} (ng*h/mL) | 31200^{b} | 27000 ± 3200 |
| λ_{z} (1/h) | 0.0008 ± 0.0002 | 0.0009 ± 0.00009 |

^{a}Median (Min-Max)
^{b}N = 2, SUBJECT 0005 not included in calculation of summary statistics

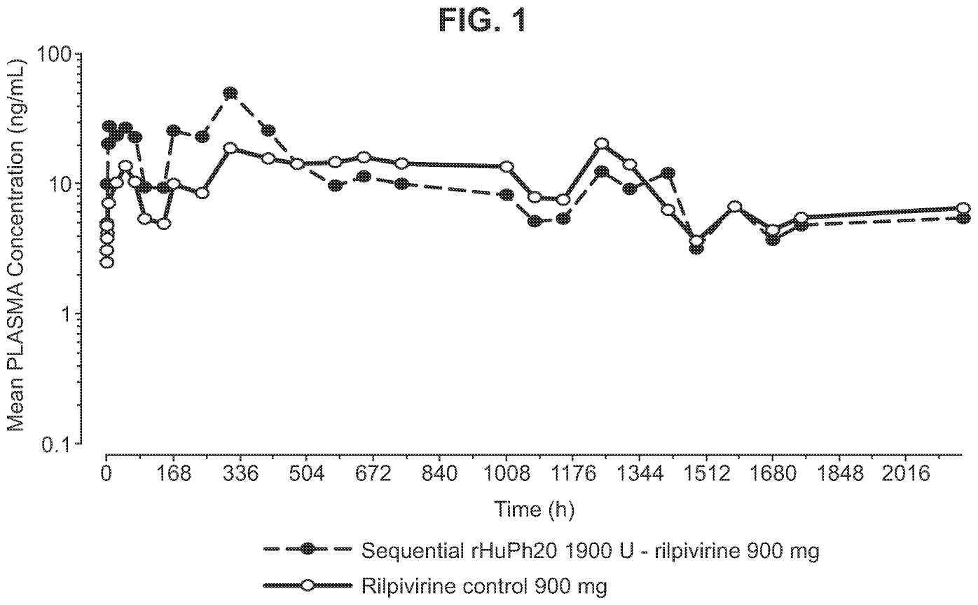

Table 1 and FIG. 1 demonstrate that administration of a hyaluronidase and a nanosuspension of rilpivirine according to the invention and administration of a nanosuspension of rilpivirine alone result in blood plasma levels of rilpivirine over a period of at least 3 months. Surprisingly a prolonged, extended, sustained release profile of rilpivirine is maintained when administered with the hyaluronidase.

### Example 2—Effects of Sequential and Admixed Administration of Rilpivirine with a Hyaluronidase Over 6 Months after Single Administration

This example compares the plasma kinetics, over a period of 6 months, for the following three conditions (i) administration of a suspension of rilpivirine (control), (ii) sequential administration of first a hyaluronidase solution then a rilpivirine suspension and (iii) admixed administration of a hyaluronidase solution and a rilpivirine suspension.

### Preparation of Rilpivirine and Hyaluronidase Compositions

(a) Suspension of Rilpivirine

The suspension of rilpivirine was prepared as described in Example 1.

(b) Solution of Hyaluronidase (rHuPH20)

The solution of hyaluronidase was prepared as described in Example 1.

Procedure

Nine minipigs with body weights ranging from 17 to 21 kg at the start of the study were used. The minipigs were fasted overnight before dosing. The minipigs were anaesthetized with propofol before dosing. Three minipigs were dosed subcutaneously in the loin with 0.44 mL of the hyaluronidase solution (10,000 U/mL) followed by 1818 mg/6.06 mL of the rilpivirine nanosuspension at the same injection site (treatment group A—sequential).

Three minipigs were dosed subcutaneously in the loin with the 1816 mg/6.5 mL admixed hyaluronidase solution (10,000 U/mL)+rilpivirine suspension (treatment group B— admixed). Three minipigs were dosed subcutaneously in the loin with the 1830 mg/6.1 mL of the control rilpivirine suspension (treatment group C—control). Vetbond 3M surgical sealant was used to seal the injection site to limit any leakage if necessary.

Method—Rilpivirine Control

The control rilpivirine suspension was prepared and administered by the following method.
1. Mix rilpivirine by horizontally shaking the container 30 times over approximately 25 cm within approximately 10 s (a back and forth arm movement=2 times). Ensure well mixed/fully re-suspended.
2. Flip-off rilpivirine vial flip cap and wipe with isopropyl alcohol wipe. Allow to dry.
3. Repeat steps 1-2 with a 2nd vial of rilpivirine. If there is a low draw, or unexpected amount of air/settling after draw, a 3rd vial may need to be prepared to ensure the proper dose level can be filled into the syringe.
4. Attach 18 G transfer needle to 10 mL syringe.
5. Invert vial and draw>3.2 mL into 10 mL syringe (or as much as can be removed from vial). Injecting 1-2 mL of air will facilitate draw.
   Repeat step 5 with the 2nd vial so that at approximately 6.5 mL of drug product is in the 10 mL syringe. Important: see note in (step 3) about preparing a 3rd vial in case of low volume draw.
6. Detach needle and attach a syringe cap to 10 mL syringe
7. Wait 5 minutes for bubbles to settle with syringe inverted.
8. Remove syringe cap, invert syringe prime off air.
9. Attach a winged infusion set.
10. Set dose at 6.1 mL after filling infusion set line until liquid forms at tip of needle (0.44 mL undeliverable/dead volume will be in infusion set).
11. Insert the winged infusion set into the subcutaneous tissue of the target injection site by pinching the skin and inserting the needle at a 30-45 degree angle.
12. Release the pinch.
13. Inject over 2 minutes at a constant rate until the syringe plunger bottoms out
14. Remove the winged infusion set and dispose.
15. Record any site leakage.

Method—(i) Sequential Administration

The sequential administration of hyaluronidase solution and then rilpivirine suspension was performed according to the following method.
1. Flip-off rHuPH20 solution vial flip cap and wipe with isopropyl alcohol wipe. Swirl vial. Allow vial stopper to dry. Attach a 18 G transfer needle to a 1 mL syringe.
2. Draw 0.70 mL into the syringe.
3. Prime the syringe and set liquid level to 0.60 mL in the syringe.
4. Remove transfer needle and attach syringe cap to 1 mL syringe.
5. Mix rilpivirine by horizontally shaking the container 30 times over approximately within approximately 10 s (a back and forth arm movement=2 times). Ensure well mixed/fully resuspended.
6. Flip-off rilpivirine vial flip cap and wipe with isopropyl alcohol wipe. Allow to dry.
7. Repeat steps 5-6 with a 2nd vial of rilpivirine. If there is a low draw, or unexpected amount of air/settling after draw, a 3rd vial may need to be prepared to ensure the proper dose level can be filled into the syringe.
8. Attach 18 G transfer needle to 10 mL syringe.
9. Invert vial and draw>3.2 mL into 10 mL syringe (or as much as can be removed from vial). Injecting 1-2 mL of air will facilitate draw.
10. Repeat step 9 with the 2nd vial so that at approximately 6.5 mL of drug product is in the 10 mL syringe. Important: see note in (step 7) about preparing a 3rd vial in case of low volume draw.
11. Detach needle and attach a syringe cap to 10 mL syringe
12. Invert syringe. Wait 5 minutes for bubbles to settle.
13. Attach a winged infusion set to the rHuPH20 1 mL syringe. Invert syringe and prime off air so that liquid forms at the tip of the needle. (0.44 mL of rHuPH20 should be in the line)
14. Insert the winged infusion set into the subcutaneous tissue of the target injection site by pinching the skin and inserting the needle at a 30-45 degree angle.
15. Release the pinch.
16. Unscrew rHuPH20 syringe from infusion set maintaining the needle in the skin. Keep open end pointed upwards so the liquid does not drain out of the infusion line while preparing the rilpivirine syringe. It is advisable to have this syringe prepared while the technician is inserting the rHuPH20 infusion line.
17. Remove the syringe cap from the 10 mL syringe with rilpivirine. Remove air and set the dose at approximately 6.5 mL.
18. Attach rilpivirine filled syringe to the open end of the infusion set.
19. Inject over 1 minute at a constant rate until the syringe plunger bottoms out (this will leave approximately 0.44 mL of rilpivirine in infusion line)
20. Remove the winged infusion set and dispose.
21. Record any site leakage.

Method—(ii) Admixed Administration

The admixed administration of hyaluronidase solution and rilpivirine suspension was performed according to the following method.
1. Flip-off rHuPH20 solution vial flip cap and wipe with isopropyl alcohol wipe. Allow to dry. Attach a 18 G transfer needle to a 1 mL syringe.
2. Draw 0.40 mL into the syringe.
3. Prime the syringe and set liquid level to 0.35 mL in the syringe.
4. Remove transfer needle and attach syringe cap to 1 mL syringe.
5. Mix rilpivirine by horizontally shaking the container 30 times over approximately within approximately 10 s (a back and forth arm movement=2 times).
   Ensure well mixed/fully resuspended.
6. Flip-off rilpivirine vial flip cap and wipe with isopropyl alcohol wipe. Allow to dry.
7. Remove syringe cap from 1 mL PH20 filled syringe and attach 25 G needle.
8. Prime the syringe so that liquid forms at needle tip and the syringe is set at approximately 0.25 mL
9. Insert 25 G needle/rHuPH20 solution syringe into vial so that the needle is in the liquid.
10. Transfer 0.25 mL of rHuPH20 solution (2500 U) into rilpivirine vial.
11. Shake vial gently.
12. Repeat steps 1-10 to prepare a second vial of rilpivirine with rHuPH20. If there is a low draw, or unexpected amount of air/settling after draw, a 3rd vial may need to be prepared to ensure the proper dose level can be filled into the syringe.
13. Attach 18 G transfer needle to 10 mL syringe.
14. Invert vial and draw>3.4 mL (or as much as can be removed from vial) into 10 mL syringe. Injecting 1-2 mL of air will facilitate draw.
15. Repeat step 14 with the 2nd prepared vial so that at approximately 7.0 mL of drug product is in the 10 mL syringe. Important: see note in (step 12) about preparing a 3rd vial in case of low volume draw.
16. Detach needle and attach a syringe cap to 10 mL syringe
17. Invert syringe and wait 5 minutes for bubbles to settle.
18. Remove syringe cap and prime off air so that a drop of liquid is at the needle, set the dose to 6.5 mL after priming.
19. Attach a winged infusion set to the 10 mL syringe. Invert syringe and prime off air so that liquid forms at the tip of the needle.
20. Insert the winged infusion set into the subcutaneous tissue of the target injection site by pinching the skin and inserting the needle at a 30-45 degree angle.
21. Inject over 1 minute at a constant rate until the syringe plunger bottoms out (this will leave approximately 0.44 mL of rilpivirine in infusion line)
22. Remove the winged infusion set and dispose.
23. Record any site leakage.

Photography of Injection Site
Injection site protrusions were assessed visually.

Blood Sampling
Blood samples of 2 mL were taken from the jugular vein from all minipigs at time intervals over the following 6 months. Blood samples were placed on EDTA. Within 1 hour of blood sampling, samples were centrifuged at 5° C. at about 1900×g for ±10 minutes to allow plasma separation. Plasma was immediately transferred into a second tube and stored in the freezer within 1 hour after the start of centrifugation. Plasma samples were analysed individually by means of a validated LC-MS/MS method.

Pharmacokinetic Data Analysis
The PK profiles of the blood plasma samples was evaluated using non-compartmental pharmacokinetic analysis (using individual Cₚ vs time profiles). Mean plasma concentrations and PK parameters (Cₘₐₓ and AUC values) were measured and the results are provided in Table 2.

Results and Discussion

PK parameters after single subcutaneous administration of rilpivirine nanosuspension at 6 mL with (sequential and admixed administration) and without rHuPH20 solution are shown in Table 2.

TABLE 2
Pharmacokinetic parameters

| | rHuPH20 + | rHuPH20 + | |
|---|---|---|---|
| | rilpivirine sequential | rilpivirine admixed | Rilpivirine |
| | (treatment | (treatment | (treatment |
| | group A) according | group B) according | group C) |
| | to the invention | to the invention | control |
| N | 3 | 3 | 3 |
| Mean Cₘₐₓ | 146 | 94 | 49^{a} |
| (ng/mL) | | | |
| Mean | 38400 | 21000 | 22000 |
| AUC_{0-1 months} | | | |
| (ng*h/mL) | | | |
| Mean | 136000 | 107000 | 78300 |
| AUC_{0-6 months} | | | |
| (ng*h/mL) | | | |

^{a}Excluding an outlier minipig (with a Cₘₐₓ of 563 ng/mL at 7 hours post-administration).

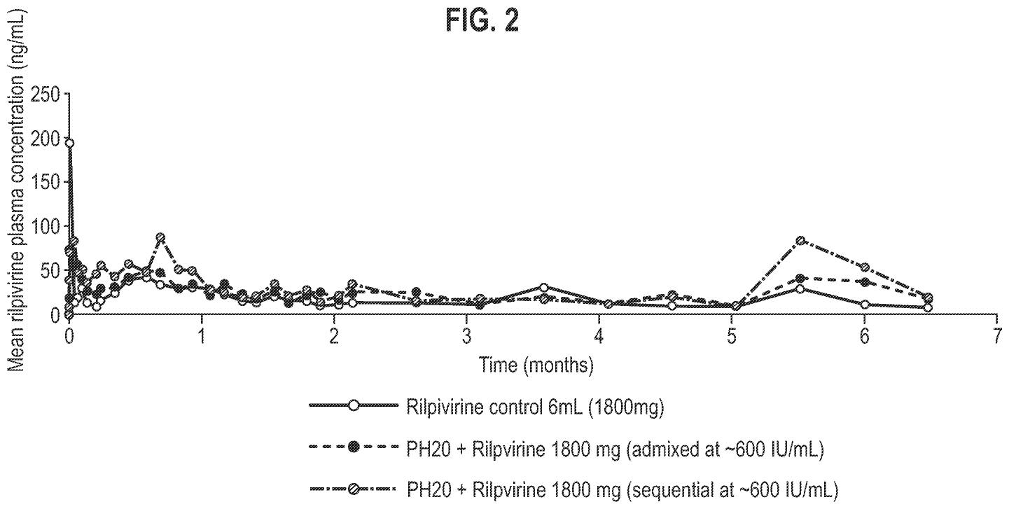

Table 2 and FIG. 2 demonstrate that both sequential and admixed administration of a hyaluronidase and a nanosuspension of rilpivirine according to the invention and administration of a nanosuspension of rilpivirine alone resulted in slow release from the injection site with measurable blood plasma levels of rilpivirine over a period of at least 6 months. Surprisingly a prolonged, extended, sustained release profile of rilpivirine is maintained when administered with the hyaluronidase both sequentially and after admixed administration.

### Example 3—Dissolution Studies with Varying Particle Size

This example compares the dissolution profile of three rilpivirine suspensions, each having a different particle size.

### Preparation of Rilpivirine Suspensions and Measurement of Particle Size

A suspension of rilpivirine was prepared according to the method described in Example 1 (suspension 1). Two further suspensions, having the same composition as Example 1 but different particle sizes, were prepared by compounding and milling (suspensions 2 and 3) as described below.

### Preparation of Suspensions 2 and 3

1. 586.62 g water for injection was added to a 2 L glass beaker containing a magnetic stir bar.
2. The correct amount of citric acid monohydrate, sodium dihydrogen phosphate monohydrate, sodium hydroxide was added and stirred until dissolved.
3. The correct amount of poloxamer 338 and glucose monohydrate was added and stirred until dissolved.
4. The diluent was filtered through a 0.22 μm filter, the beaker was rinsed with the remaining 100 mL water for injection and filtered.
5. Rilpivirine microfine was added and stirred until a homogenous suspension was obtained.
6. 500 mL of the suspension was transferred in sterilized beaker and placed in a double walled cooled glass beaker with magnetic stir bar.
7. Start milling on Netzsch Labstar, mill until target particle size distribution is reached. For suspension 2, milling time was about 180 minutes. For suspension 3, milling time was about 35 minutes.
8. The particle size distribution was measured during milling.
9. Each suspension was diluted to 300 mg/mL.

Particle Size Distribution Measurement

The volume-based particle size distribution of the rilpivirine suspensions was determined by means of wet dispersion laser diffraction, using a Malvern Mastersizer 3000 laser diffraction (Malvern Instruments) and Hydro MV wet dispersion module.

The particle size of the three rilpivirine suspensions were as defined in Table 3.

TABLE 3
Particle sizes

| Suspension | Dᵥ50 (μm) | Dᵥ90 (μm) |
|---|---|---|
| 1 | 0.29 | 0.69 |
| 2 | 0.39 | 1.91 |
| 3 | 2.46 | 5.55 |

In Vitro Dissolution Measurement

The dissolution of the three rilpivirine suspensions in water was performed using Paddle Apparatus (USP type 2, Ph.Eur., JP.) at 50 rpm in 900 mL of 6.0% w/v Polysorbate 20 in 0.05 M Sodium Phosphate buffer pH 7.4, at 5.0±0.5° C. An amount of 64.98 mg (=0.06 mL×1.083 g/mL (the theoretical density of the suspension)) ±5% of homogeneous suspension of rilpivirine (corresponding to 18±0.9 mg of rilpivirine) was added.

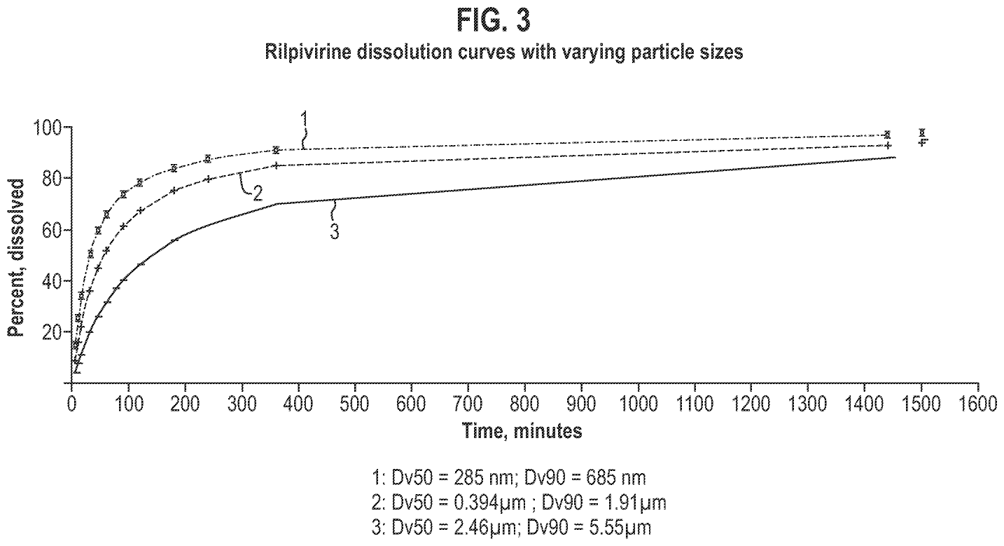

The determination of the quantity of rilpivirine present in the dissolution samples is based upon a gradient ultra-high performance liquid chromatographic (UHPLC) method with UV detection at 280 nm. Results are shown in FIG. 3.

Results and Discussion

FIG. 3 demonstrates that administration of rilpivirine in the form of micro- or nanoparticles having larger particle sizes as shown in Table 3 surprisingly lowered, i.e. flattened, the dissolution profile of rilpivirine.

### Example 4—Further Dissolution Studies with Varying Particle Size

This example compares the dissolution profile of five rilpivirine suspensions, each having a different particle size.

### Preparation of Rilpivirine Suspensions and Measurement of Particle Size

Five suspensions of rilpivirine were prepared according to a method corresponding to the method described for suspensions 2 and 3 in Example 3. The volume-based particle size distribution of the rilpivirine micro- or nanoparticles in suspension was determined according to a method corresponding to the method that is specified in Example 3.

TABLE 4
Particle sizes

| Suspension | Dᵥ50 (μm) | Dᵥ90 (μm) |
|---|---|---|
| 1 | 0.42 | 2.12 |
| 2 | 0.63 | 2.85 |
| 3 | 1.29 | 3.69 |
| 4 | 1.99 | 5.00 |
| 5 | 2.72 | 6.46 |

In Vitro Dissolution Measurement

The dissolution of the five rilpivirine suspensions in water was performed according to the method that is specified in Example 3.

Results and Discussion

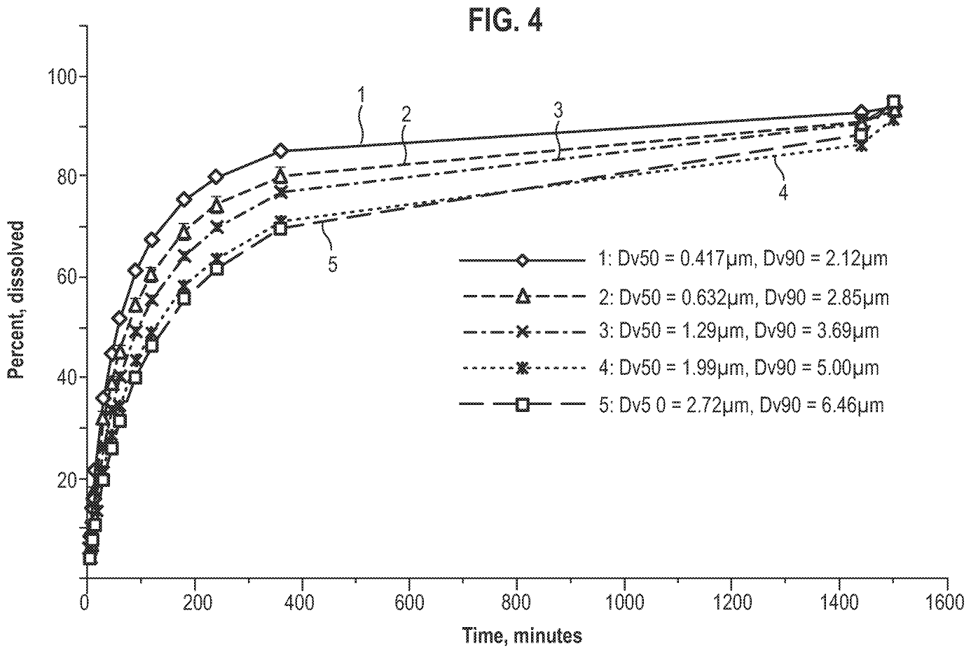

FIG. 4 and Table 4 demonstrate that as the particle size of rilpivirine in the form of micro- or nanoparticles is increased the dissolution profile of rilpivirine is lowered, i.e. flattened.

Also described herein are the following numbered clauses.
1. Rilpivirine or a pharmaceutically acceptable salt thereof and a hyaluronidase for use in the treatment or prevention of HIV infection in a subject,
   wherein the rilpivirine or a pharmaceutically acceptable salt thereof is in the form of micro- or nanoparticles in suspension,
   wherein the rilpivirine or pharmaceutically acceptable salt thereof and hyaluronidase are administered to the subject by subcutaneous or intramuscular injection, and
   wherein the rilpivirine or pharmaceutically acceptable salt thereof and hyaluronidase are administered intermittently at a time interval of about three months to about two years.
2. The rilpivirine or a pharmaceutically acceptable salt thereof and a hyaluronidase for use according to clause 1, wherein the hyaluronidase is recombinant human hyaluronidase (e.g. rHuPH20 enzyme), for example, comprising the amino acid sequence of SEQ ID NO: 1.
3. The rilpivirine or a pharmaceutically acceptable salt thereof and a hyaluronidase for use according to any one of the preceding clauses, wherein the time interval is about three months to about one year.
4. The rilpivirine or a pharmaceutically acceptable salt thereof and a hyaluronidase for use according to clause 3, wherein the time interval is about three months to about six months.
5. The rilpivirine or a pharmaceutically acceptable salt thereof and a hyaluronidase for use according to clause 3, wherein the time interval is about six months to about one year, in particular wherein the time interval is about six months.
6. The rilpivirine or a pharmaceutically acceptable salt thereof and a hyaluronidase for use according to any one of the preceding clauses, wherein the rilpivirine or a pharmaceutically acceptable salt thereof and hyaluronidase are administered simultaneously or sequentially.
7. The rilpivirine or a pharmaceutically acceptable salt thereof and a hyaluronidase for use according to any one of the preceding clauses, wherein the micro- or nanoparticles have a surface modifier adsorbed to their surface.
8. The rilpivirine or a pharmaceutically acceptable salt thereof and a hyaluronidase for use according to clause 7, wherein the surface modifier is a poloxamer.
9. The rilpivirine or a pharmaceutically acceptable salt thereof and a hyaluronidase for use according to clause 8, wherein the poloxamer is poloxamer 338.
10. The rilpivirine or a pharmaceutically acceptable salt thereof and a hyaluronidase for use according to any one of the preceding clauses, wherein the average effective particle size of the micro- or nanoparticles is less than about 1 μm.
11. The rilpivirine or a pharmaceutically acceptable salt thereof and a hyaluronidase for use according to clause 10, wherein the average effective particle size of the micro- or nanoparticles is less than about 500 nm.
12. The rilpivirine or a pharmaceutically acceptable salt thereof and a hyaluronidase for use according to clause 11, wherein the average effective particle size of the micro- or nanoparticles is from about 100 nm to about 300 nm.
13. The rilpivirine or a pharmaceutically acceptable salt thereof and a hyaluronidase for use according to clause 12, wherein the average effective particle size of the micro- or nanoparticles is from about 150 nm to about 250 nm.
14. The rilpivirine or a pharmaceutically acceptable salt thereof and a hyaluronidase for use according to clause 13, wherein the average effective particle size of the micro- or nanoparticles is from about 180 nm to about 220 nm.
15. The rilpivirine or a pharmaceutically acceptable salt thereof and a hyaluronidase for use according to any one of the preceding clauses, wherein the rilpivirine or a pharmaceutically acceptable salt thereof and the hyaluronidase are administered sequentially.
16. The rilpivirine or a pharmaceutically acceptable salt thereof and a hyaluronidase for use according to any one of the preceding clauses, wherein the rilpivirine or a pharmaceutically acceptable salt thereof and hyaluronidase are administered in separate pharmaceutical compositions.
17. The rilpivirine or a pharmaceutically acceptable salt thereof and a hyaluronidase for use according to clause 16, wherein the pharmaceutical composition comprising the hyaluronidase is a solution, and the concentration of the hyaluronidase in the solution is from about 50 to about 10,000 U/mL, in particular is about 2,000 U/mL.
18. The rilpivirine or a pharmaceutically acceptable salt thereof and a hyaluronidase for use according to any one of clauses 1-14, wherein the rilpivirine or a pharmaceutically acceptable salt thereof and the hyaluronidase are administered as a combined pharmaceutical composition.
19. The rilpivirine or a pharmaceutically acceptable salt thereof and a hyaluronidase for use according to any one of the preceding clauses, wherein the rilpivirine or a pharmaceutically acceptable salt thereof and the hyaluronidase are administered by subcutaneous injection.
20. The rilpivirine or a pharmaceutically acceptable salt thereof and a hyaluronidase for use according to any one of the preceding clauses, wherein the suspension comprises a pharmaceutically acceptable aqueous carrier in which the rilpivirine or a pharmaceutically acceptable salt thereof is suspended.
21. The rilpivirine or a pharmaceutically acceptable salt thereof and a hyaluronidase for use according to any one of the preceding clauses, wherein the treatment or prevention of HIV infection is treatment of HIV infection.
22. The rilpivirine or a pharmaceutically acceptable salt thereof and a hyaluronidase for use according to clause 21, wherein each administration of the rilpivirine or a pharmaceutically acceptable salt thereof comprises from about 2700 mg to about 5400 mg of rilpivirine or a pharmaceutically acceptable salt thereof, in particular from about 2700 mg to about 4500 mg of rilpivirine or a pharmaceutically acceptable salt thereof.
23. The rilpivirine or a pharmaceutically acceptable salt thereof and a hyaluronidase for use according to any one of the preceding clauses, wherein the HIV infection is HIV type 1 (HIV-1) infection.
24. The rilpivirine or a pharmaceutically acceptable salt thereof and a hyaluronidase for use according to any one of the preceding clauses, wherein the subject is a human.
25. The rilpivirine or a pharmaceutically acceptable salt thereof and a hyaluronidase for use according to any one of the preceding clauses, wherein the rilpivirine or a pharmaceutically acceptable salt thereof is rilpivirine.
26. A combination for use in the treatment or prevention of HIV infection, wherein the combination comprises rilpivirine or a pharmaceutically acceptable salt thereof and a hyaluronidase, wherein the rilpivirine or pharmaceutically acceptable salt thereof is in the form of micro- or nanoparticles in suspension, and wherein the combination is administered intermittently by subcutaneous or intramuscular injection at a time interval of about three months to about two years.
27. The combination for use according to clause 26, wherein the hyaluronidase is recombinant human hyaluronidase (e.g. rHuPH20), for example, comprising the amino acid sequence of SEQ ID NO: 1.
28. The combination for use according to any one of clauses 26-27, wherein the time interval is about three months to about one year.
29. The combination for use according to clause 28, wherein the time interval is about three months to about six months.
30. The combination for use according to clause 28, wherein the time interval is about six months to about one year, in particular wherein the time interval is about six months.
31. The combination for use according to any one of clauses 26-30, wherein the rilpivirine or a pharmaceutically acceptable salt thereof and hyaluronidase are administered simultaneously or sequentially.
32. The combination for use according to any one of clauses 26-31, wherein the micro- or nanoparticles have a surface modifier adsorbed to their surface.
33. The combination for use according to clause 32, wherein the surface modifier is a poloxamer.
34. The combination for use according to clause 33, wherein the poloxamer is poloxamer 338.
35. The combination for use according to any one of clauses 26-34, wherein the average effective particle size of the micro- or nanoparticles is less than about 1 μm.
36. The combination for use according to clause 35, wherein the average effective particle size of the micro- or nanoparticles is less than about 500 nm.
37. The combination for use according to clause 36, wherein the average effective particle size of the micro- or nanoparticles is from about 100 nm to about 300 nm.
38. The combination for use according to clause 37, wherein the average effective particle size of the micro- or nanoparticles is from about 150 nm to about 250 nm.
39. The combination for use according to clause 38, wherein the average effective particle size of the micro- or nanoparticles is from about 180 nm to about 220 nm.
40. The combination for use according to any one of clauses 26-39, wherein the rilpivirine or a pharmaceutically acceptable salt thereof and the hyaluronidase are administered sequentially.
41. The combination for use according to any one of clauses 26-40, wherein the rilpivirine or a pharmaceutically acceptable salt thereof and hyaluronidase are administered in separate pharmaceutical compositions.
42. The combination for use according to clause 41, wherein the pharmaceutical composition comprising the hyaluronidase is a solution, and the concentration of the hyaluronidase in the solution is from about 50 to about 10,000 U/mL, in particular is about 2,000 U/mL.
43. The combination for use according to any one of clauses 26-39, wherein the rilpivirine or a pharmaceutically acceptable salt thereof and the hyaluronidase are administered as a combined pharmaceutical composition.
44. The combination for use according to any one of clauses 26-43, wherein the rilpivirine or a pharmaceutically acceptable salt thereof and the hyaluronidase are administered by subcutaneous injection.
45. The combination for use according to any one of clauses 26-44, wherein the suspension comprises a pharmaceutically acceptable aqueous carrier in which the rilpivirine or a pharmaceutically acceptable salt thereof is suspended.
46. The combination for use according to any one of clauses 26-45, wherein the treatment or prevention of HIV infection is treatment of HIV infection.
47. The combination for use according to clause 46, wherein each administration of the rilpivirine or a pharmaceutically acceptable salt thereof comprises from about 2700 mg to about 5400 mg of rilpivirine or a pharmaceutically acceptable salt thereof, in particular from about 2700 mg to about 4500 mg of rilpivirine or a pharmaceutically acceptable salt thereof.
48. The combination for use according to any one of clauses 26-47, wherein the HIV infection is HIV type 1 (HIV-1) infection.
49. The combination for use according to any one of clauses 26-48, wherein the subject is a human.
50. The combination for use according to any one of clauses 26-49, wherein the rilpivirine or a pharmaceutically acceptable salt thereof is rilpivirine.
51. Products containing rilpivirine or a pharmaceutically acceptable salt thereof and a hyaluronidase as a combined preparation for simultaneous or sequential use in the treatment or prevention of HIV infection by subcutaneous or intramuscular injection,
   wherein the rilpivirine or pharmaceutically acceptable salt thereof is in the form of micro- or nanoparticles in suspension, and
   wherein the rilpivirine or pharmaceutically acceptable salt thereof and the hyaluronidase are administered intermittently at a time interval of about three months to about two years.
52. The products for simultaneous or sequential use according to clause 51, wherein the hyaluronidase is recombinant human hyaluronidase (e.g. rHuPH20), for example, comprising the amino acid sequence of SEQ ID NO: 1.
53. The products for simultaneous or sequential use according to any one of clauses 51-52, wherein the time interval is about three months to about one year.
54. The products for simultaneous or sequential use according to clause 53, wherein the time interval is about three months to about six months.
55. The products for simultaneous or sequential use according to clause 53, wherein the time interval is about six months to about one year, in particular wherein the time interval is about six months.
56. The products for simultaneous or sequential use according to any one of clauses 51-55, wherein the rilpivirine or a pharmaceutically acceptable salt thereof and hyaluronidase are administered sequentially.
57. The products for simultaneous or sequential use according to any one of clauses 51-56, wherein the micro- or nanoparticles have a surface modifier adsorbed to their surface.
58. The products for simultaneous or sequential use according to clause 57, wherein the surface modifier is a poloxamer.
59. The products for simultaneous or sequential use according to clause 58, wherein the poloxamer is poloxamer 338.
60. The products for simultaneous or sequential use according to any one of clauses 51-59, wherein the average effective particle size of the micro- or nanoparticles is less than about 1 μm.
61. The products for simultaneous or sequential use according to clause 60, wherein the average effective particle size of the micro- or nanoparticles is less than about 500 nm.
62. The products for simultaneous or sequential use according to clause 61, wherein the average effective particle size of the micro- or nanoparticles is from about 100 nm to about 300 nm.
63. The products for simultaneous or sequential use according to clause 62, wherein the average effective particle size of the micro- or nanoparticles is from about 150 nm to about 250 nm.
64. The products for simultaneous or sequential use according to clause 63, wherein the average effective particle size of the micro- or nanoparticles is from about 180 nm to about 220 nm.
65. The products for simultaneous or sequential use according to any one of clauses 51-64, wherein the rilpivirine or a pharmaceutically acceptable salt thereof and the hyaluronidase are administered sequentially by subcutaneous injection.
66. The products for simultaneous or sequential use according to any one of clauses 51-65, wherein the rilpivirine or a pharmaceutically acceptable salt thereof and hyaluronidase are administered in separate pharmaceutical compositions.
67. The products for simultaneous or sequential use according to clause 66, wherein the pharmaceutical composition comprising the hyaluronidase is a solution, and the concentration of the hyaluronidase in the solution is from about 50 to about 10,000 U/mL, in particular is about 2,000 U/mL.
68. The products for simultaneous or sequential use according to any one of clauses 51-64, wherein the rilpivirine or a pharmaceutically acceptable salt thereof and the hyaluronidase are administered as a combined pharmaceutical composition.
69. The products for simultaneous or sequential use according to any one of clauses 51-68, wherein the rilpivirine or a pharmaceutically acceptable salt thereof and the hyaluronidase are administered by subcutaneous injection.
70. The products for simultaneous or sequential use according to any one of clauses 51-69, wherein the suspension comprises a pharmaceutically acceptable aqueous carrier in which the rilpivirine or a pharmaceutically acceptable salt thereof is suspended.
71. The products for simultaneous or sequential use according to any one of clauses 51-70, wherein the treatment or prevention of HIV infection is treatment of HIV infection.
72. The products for simultaneous or sequential use according to clause 71, wherein each administration of the rilpivirine or a pharmaceutically acceptable salt thereof comprises from about 2700 mg to about 5400 mg of rilpivirine or a pharmaceutically acceptable salt thereof, in particular from about 2700 mg to about 4500 mg of rilpivirine or a pharmaceutically acceptable salt thereof.
73. The products for simultaneous or sequential use according to any one of clauses 51-72, wherein the HIV infection is HIV type 1 (HIV-1) infection.
74. The products for simultaneous or sequential use according to any one of clauses 51-73, wherein the subject is a human.

The products for simultaneous or sequential use according to any one of clauses 51-74, wherein the rilpivirine or a pharmaceutically acceptable salt thereof is rilpivirine.
76. A kit of parts comprising rilpivirine or a pharmaceutically acceptable salt thereof and a hyaluronidase for simultaneous or sequential use in the treatment or prevention of HIV infection by subcutaneous or intramuscular injection, wherein the rilpivirine or a pharmaceutically acceptable salt thereof is in the form of micro- or nanoparticles in suspension, and wherein the rilpivirine or a pharmaceutically acceptable salt thereof and the hyaluronidase are administered intermittently at a time interval of about three months to about two years.
77. The kit of parts for simultaneous or sequential use according to clause 76, wherein the hyaluronidase is recombinant human hyaluronidase (e.g. rHuPH20), for example, comprising the amino acid sequence of SEQ ID NO: 1.
78. The kit of parts for simultaneous or sequential use according to any one of clauses 76-77, wherein the time interval is about three months to about one year.
79. The kit of parts for simultaneous or sequential use according to clause 78, wherein the time interval is about three months to about six months.
80. The kit of parts for simultaneous or sequential use according to clause 78, wherein the time interval is about six months to about one year, in particular wherein the time interval is about six months.
81. The kit of parts for simultaneous or sequential use according to any one of clauses 76-80, wherein the rilpivirine or a pharmaceutically acceptable salt thereof and hyaluronidase are administered sequentially.
82. The kit of parts for simultaneous or sequential use according to any one of clauses 76-81, wherein the micro- or nanoparticles have a surface modifier adsorbed to their surface.
83. The kit of parts for simultaneous or sequential use according to clause 82, wherein the surface modifier is a poloxamer.
84. The kit of parts for simultaneous or sequential use according to clause 83, wherein the poloxamer is poloxamer 338.
85. The kit of parts for simultaneous or sequential use according to any one of clauses 76-84, wherein the average effective particle size of the micro- or nanoparticles is less than about 1 μm.
86. The kit of parts for simultaneous or sequential use according to clause 85, wherein the average effective particle size of the micro- or nanoparticles is less than about 500 nm.
87. The kit of parts for simultaneous or sequential use according to clause 86, wherein the average effective particle size of the micro- or nanoparticles is from about 100 nm to about 300 nm.
88. The kit of parts for simultaneous or sequential use according to clause 87, wherein the average effective particle size of the micro- or nanoparticles is from about 150 nm to about 250 nm.
89. The kit of parts for simultaneous or sequential use according to clause 88, wherein the average effective particle size of the micro- or nanoparticles is from about 180 nm to about 220 nm.
90. The kit of parts for simultaneous or sequential use according to any one of clauses 76-89, wherein the rilpivirine or a pharmaceutically acceptable salt thereof and the hyaluronidase are administered sequentially by subcutaneous injection.
91. The kit of parts for simultaneous or sequential use according to any one of clauses 76-90, wherein the rilpivirine or a pharmaceutically acceptable salt thereof and hyaluronidase are administered in separate pharmaceutical compositions.
92. The kit of parts for simultaneous or sequential use according to clause 91, wherein the pharmaceutical composition comprising the hyaluronidase is a solution, and the concentration of the hyaluronidase in the solution is from about 50 to about 10,000 U/mL, in particular is about 2,000 U/mL.
93. The kit of parts for simultaneous or sequential use according to any one of clauses 76-89, wherein the rilpivirine or a pharmaceutically acceptable salt thereof and the hyaluronidase are administered as a combined pharmaceutical composition.
94. The kit of parts for simultaneous or sequential use according to any one of clauses 76-93, wherein the rilpivirine or a pharmaceutically acceptable salt thereof and the hyaluronidase are administered by subcutaneous injection.
95. The kit of parts for simultaneous or sequential use according to any one of clauses 76-94, wherein the suspension comprises a pharmaceutically acceptable aqueous carrier in which the rilpivirine or a pharmaceutically acceptable salt thereof is suspended.
96. The kit of parts for simultaneous or sequential use according to any one of clauses 76-95, wherein the treatment or prevention of HIV infection is treatment of HIV infection.
97. The kit of parts for simultaneous or sequential use according to clause 96, wherein each administration of the rilpivirine ora pharmaceutically acceptable salt thereof comprises from about 2700 mg to about 5400 mg of rilpivirine or a pharmaceutically acceptable salt thereof, in particular from about 2700 mg to about 4500 mg of rilpivirine or a pharmaceutically acceptable salt thereof.
98. The kit of parts for simultaneous or sequential use according to any one of clauses 76-97, wherein the HIV infection is HIV type 1 (HIV-1) infection.
99. The kit of parts for simultaneous or sequential use according to any one of clauses 76-98, wherein the subject is a human.
100. The kit of parts for simultaneous or sequential use according to any one of clauses 76-99, wherein the rilpivirine or a pharmaceutically acceptable salt thereof is rilpivirine.
101. Rilpivirine or a pharmaceutically acceptable salt thereof in the form of micro- or nanoparticles in suspension for use in the treatment or prevention of HIV infection by subcutaneous or intramuscular injection, wherein the rilpivirine or pharmaceutically acceptable salt thereof is administered in combination with a hyaluronidase that is administered by subcutaneous or intramuscular injection, and wherein the rilpivirine or pharmaceutically acceptable salt thereof and the hyaluronidase are administered intermittently at a time interval of about three months to about two years.
102. The rilpivirine or a pharmaceutically acceptable salt thereof for use according to clause 101, wherein the hyaluronidase is recombinant human hyaluronidase (e.g. rHuPH20), for example, comprising the amino acid sequence of SEQ ID NO: 1.
103. The rilpivirine or a pharmaceutically acceptable salt thereof for use according to any one of clauses 101-102, wherein the time interval is about three months to about one year.
104. The rilpivirine or a pharmaceutically acceptable salt thereof for use according to clause 103, wherein the time interval is about three months to about six months, in particular wherein the time interval is about six months.
105. The rilpivirine or a pharmaceutically acceptable salt thereof for use according to clause 103, wherein the time interval is about six months to about one year.
106. The rilpivirine or a pharmaceutically acceptable salt thereof for use according to any one of clauses 101-105, wherein the rilpivirine ora pharmaceutically acceptable salt thereof and hyaluronidase are administered simultaneously or sequentially.
107. The rilpivirine or a pharmaceutically acceptable salt thereof for use according to any one of clauses 101-106, wherein the micro- or nanoparticles have a surface modifier adsorbed to their surface.
108. The rilpivirine or a pharmaceutically acceptable salt thereof for use according to clause 107, wherein the surface modifier is a poloxamer.
109. The rilpivirine or a pharmaceutically acceptable salt thereof for use according to clause 108, wherein the poloxamer is poloxamer 338.
110. The rilpivirine or a pharmaceutically acceptable salt thereof for use according to any one of clauses 101-109, wherein the average effective particle size of the micro- or nanoparticles is less than about 1 μm.
111. The rilpivirine or a pharmaceutically acceptable salt thereof for use according to clause 110, wherein the average effective particle size of the micro- or nanoparticles is less than about 500 nm.
112. The rilpivirine or a pharmaceutically acceptable salt thereof for use according to clause 111, wherein the average effective particle size of the micro- or nanoparticles is from about 100 nm to about 300 nm.
113. The rilpivirine or a pharmaceutically acceptable salt thereof for use according to clause 112, wherein the average effective particle size of the micro- or nanoparticles is from about 150 nm to about 250 nm.
114. The rilpivirine or a pharmaceutically acceptable salt thereof for use according to clause 113, wherein the average effective particle size of the micro- or nanoparticles is from about 180 nm to about 220 nm.
115. The rilpivirine or a pharmaceutically acceptable salt thereof for use according to any one of clauses 101-114, wherein the rilpivirine or a pharmaceutically acceptable salt thereof and the hyaluronidase are administered sequentially.
116. The rilpivirine or a pharmaceutically acceptable salt thereof for use according to any one of clauses 101-115, wherein the rilpivirine or a pharmaceutically acceptable salt thereof and hyaluronidase are administered in separate pharmaceutical compositions.
117. The rilpivirine or a pharmaceutically acceptable salt thereof for use according to clause 116, wherein the pharmaceutical composition comprising the hyaluronidase is a solution, and the concentration of the hyaluronidase in the solution is from about 50 to about 10,000 U/mL, in particular is about 2,000 U/mL.
118. The rilpivirine or a pharmaceutically acceptable salt thereof for use according to any one of clauses 101-114, wherein the rilpivirine or a pharmaceutically acceptable salt thereof and the hyaluronidase are administered as a combined pharmaceutical composition.
119. The rilpivirine or a pharmaceutically acceptable salt thereof for use according to any one of clauses 101-118, wherein the rilpivirine or a pharmaceutically acceptable salt thereof and the hyaluronidase are administered by subcutaneous injection.
120. The rilpivirine or a pharmaceutically acceptable salt thereof for use according to any one of clauses 101-119, wherein the suspension comprises a pharmaceutically acceptable aqueous carrier in which the rilpivirine or a pharmaceutically acceptable salt thereof is suspended.
121. The rilpivirine or a pharmaceutically acceptable salt thereof for use according to any one of clauses 101-120, wherein the treatment or prevention of HIV infection is treatment of HIV infection.
122. The rilpivirine or a pharmaceutically acceptable salt thereof for use according to clause 121, wherein each administration of the rilpivirine or a pharmaceutically acceptable salt thereof comprises from about 2700 mg to about 5400 mg of rilpivirine or a pharmaceutically acceptable salt thereof, in particular from about 2700 mg to about 4500 mg of rilpivirine or a pharmaceutically acceptable salt thereof.
123. The rilpivirine or a pharmaceutically acceptable salt thereof for use according to any one of clauses 101-122, wherein the HIV infection is HIV type 1 (HIV-1) infection.
124. The rilpivirine or a pharmaceutically acceptable salt thereof for use according to any one of clauses 101-123, wherein the subject is a human.
125. The rilpivirine or a pharmaceutically acceptable salt thereof for use according to any one of clauses 101-124, wherein the rilpivirine ora pharmaceutically acceptable salt thereof is rilpivirine.
126. Use of rilpivirine or a pharmaceutically acceptable salt thereof for the manufacture of a medicament for treating or preventing HIV infection in a subject,
   wherein the rilpivirine or pharmaceutically acceptable salt thereof is in the form of micro- or nanoparticles in suspension and is administered in combination with a hyaluronidase,
   wherein the rilpivirine or pharmaceutically acceptable salt thereof and the hyaluronidase are administered to the subject by subcutaneous or intramuscular injection, and
   wherein the rilpivirine or pharmaceutically acceptable salt thereof and the hyaluronidase are administered intermittently at a time interval of about three months to about two years.
127. The use according to clause 126, wherein the hyaluronidase is recombinant human hyaluronidase (e.g. rHuPH20), for example, comprising the amino acid sequence of SEQ ID NO: 1.
128. The use according to any one of clauses 126-127, wherein the time interval is about three months to about one year.
129. The use according to clause 128, wherein the time interval is about three months to about six months.
130. The use according to clause 129, wherein the time interval is about six months to about one year, in particular wherein the time interval is about six months.
131. The use according to any one of clauses 126-130, wherein the rilpivirine or a pharmaceutically acceptable salt thereof and hyaluronidase are administered simultaneously or sequentially.
132. The use according to any one of clauses 126-131, wherein the micro- or nanoparticles have a surface modifier adsorbed to their surface.
133. The use according to clause 132, wherein the surface modifier is a poloxamer.
134. The use according to clause 133, wherein the poloxamer is poloxamer 338.
135. The use according to any one of clauses 126-134, wherein the average effective particle size of the micro- or nanoparticles is less than about 1 μm.
136. The use according to clause 135, wherein the average effective particle size of the micro- or nanoparticles is less than about 500 nm.
137. The use according to clause 136, wherein the average effective particle size of the micro- or nanoparticles is from about 100 nm to about 300 nm.
138. The use according to clause 137, wherein the average effective particle size of the micro- or nanoparticles is from about 150 nm to about 250 nm.
139. The use according to clause 138, wherein the average effective particle size of the micro- or nanoparticles is from about 180 nm to about 220 nm.
140. The use according to any one of clauses 126-139, wherein the rilpivirine or a pharmaceutically acceptable salt thereof and the hyaluronidase are administered sequentially.
141. The use according to any one of clauses 126-140, wherein the rilpivirine or a pharmaceutically acceptable salt thereof and hyaluronidase are administered in separate pharmaceutical compositions.
142. The use according to clause 141, wherein the pharmaceutical composition comprising the hyaluronidase is a solution, and the concentration of the hyaluronidase in the solution is from about 50 to about 10,000 U/mL, in particular is about 2,000 U/mL.
143. The use according to any one of clauses 126-139, wherein the rilpivirine or a pharmaceutically acceptable salt thereof and the hyaluronidase are administered as a combined pharmaceutical composition.
144. The use according to any one of clauses 126-143, wherein the rilpivirine or a pharmaceutically acceptable salt thereof and the hyaluronidase are administered by subcutaneous injection.
145. The use according to any one of clauses 126-144, wherein the suspension comprises a pharmaceutically acceptable aqueous carrier in which the rilpivirine or a pharmaceutically acceptable salt thereof is suspended.
146. The use according to any one of clauses 126-145, wherein the use is for the manufacture of a medicament for treating HIV infection in a subject.
147. The use according to clause 146, wherein each administration of the rilpivirine or a pharmaceutically acceptable salt thereof comprises from about 2700 mg to about 5400 mg of rilpivirine or a pharmaceutically acceptable salt thereof, in particular from about 2700 mg to about 4500 mg of rilpivirine or a pharmaceutically acceptable salt thereof.
148. The use according to any one of clauses 126-147, wherein the HIV infection is HIV type 1 (HIV-1) infection.
149. The use according to any one of clauses 126-148, wherein the subject is a human.
150. The use according to any one of clauses 126-149, wherein the rilpivirine or a pharmaceutically acceptable salt thereof is rilpivirine.
151. A combination comprising rilpivirine or a pharmaceutically acceptable salt thereof and a hyaluronidase, wherein the rilpivirine or a pharmaceutically acceptable salt thereof is in the form of micro- or nanoparticles in suspension.
152. The combination according to clause 151, wherein the hyaluronidase is recombinant human hyaluronidase (e.g. rHuPH20), for example, comprising the amino acid sequence of SEQ ID NO: 1.
153. The combination according to any one of clauses 151-152, wherein the rilpivirine or a pharmaceutically acceptable salt thereof and hyaluronidase are formulated for simultaneous or sequential administration.
154. The combination according to any one of clauses 151-153, wherein the micro- or nanoparticles have a surface modifier adsorbed to their surface.
155. The combination according to clause 154, wherein the surface modifier is a poloxamer.
156. The combination according to clause 155, wherein the poloxamer is poloxamer 338.
157. The combination according to any one of clauses 151-156, wherein the average effective particle size of the micro- or nanoparticles is less than about 1 μm.
158. The combination according to clause 157, wherein the average effective particle size of the micro- or nanoparticles is less than about 500 nm.
159. The combination according to clause 158, wherein the average effective particle size of the micro- or nanoparticles is from about 100 nm to about 300 nm.
160. The combination according to clause 159, wherein the average effective particle size of the micro- or nanoparticles is from about 150 nm to about 250 nm.
161. The combination according to clause 160, wherein the average effective particle size of the micro- or nanoparticles is from about 180 nm to about 220 nm.
162. The combination according to any one of clauses 151-161, wherein the rilpivirine or a pharmaceutically acceptable salt thereof and the hyaluronidase are formulated for sequential administration.
163. The combination according to any one of clauses 151-162, wherein the rilpivirine or a pharmaceutically acceptable salt thereof and/or hyaluronidase are/is formulated for administration in separate pharmaceutical compositions.
164. The combination according to clause 163, wherein the pharmaceutical composition comprising the hyaluronidase is a solution, and the concentration of the hyaluronidase in the solution is from about 50 to about 10,000 U/mL, in particular is about 2,000 U/mL.
165. The combination according to any one of clauses 151-161, wherein the rilpivirine or a pharmaceutically acceptable salt thereof and the hyaluronidase are formulated for administration as a combined pharmaceutical composition.
166. The combination according to any one of clauses 151-165, wherein the rilpivirine or a pharmaceutically acceptable salt thereof and the hyaluronidase are formulated for administration by subcutaneous injection.
167. The combination according to any one of clauses 151-166, wherein the suspension comprises a pharmaceutically acceptable aqueous carrier in which the rilpivirine or a pharmaceutically acceptable salt thereof is suspended.
168. The combination according to any one of clauses 151-167, wherein the rilpivirine or a pharmaceutically acceptable salt thereof is rilpivirine.
169. A kit of parts comprising rilpivirine or a pharmaceutically acceptable salt thereof and a hyaluronidase, wherein the rilpivirine or a pharmaceutically acceptable salt thereof is in the form of micro- or nanoparticles in suspension.
170. The kit of parts according to clause 169, wherein the hyaluronidase is recombinant human hyaluronidase (e.g. rHuPH20), for example, comprising the amino acid sequence of SEQ ID NO: 1.
171. The kit of parts according to any one of clauses 169-170 wherein the rilpivirine or a pharmaceutically acceptable salt thereof and hyaluronidase are formulated for simultaneous or sequential administration.
172. The kit of parts according to any one of clauses 169-171, wherein the micro- or nanoparticles have a surface modifier adsorbed to their surface.
173. The kit of parts according to clause 172, wherein the surface modifier is a poloxamer.
174. The kit of parts according to clause 173, wherein the poloxamer is poloxamer 338.
175. The kit of parts according to any one of clauses 169-174, wherein the average effective particle size of the micro- or nanoparticles is less than about 1 μm.
176. The kit of parts according to clause 175, wherein the average effective particle size of the micro- or nanoparticles is less than about 500 nm.
177. The kit of parts according to clause 176, wherein the average effective particle size of the micro- or nanoparticles is from about 100 nm to about 300 nm.
178. The kit of parts according to clause 177, wherein the average effective particle size of the micro- or nanoparticles is from about 150 nm to about 250 nm.
179. The kit of parts according to clause 178, wherein the average effective particle size of the micro- or nanoparticles is from about 180 nm to about 220 nm.
180. The kit of parts according to any one of clauses 169-179, wherein the rilpivirine or a pharmaceutically acceptable salt thereof and the hyaluronidase are formulated for sequential administration.
181. The kit of parts according to any one of clauses 169-180, wherein the rilpivirine or a pharmaceutically acceptable salt thereof and/or hyaluronidase are/is formulated for administration in separate pharmaceutical compositions.
182. The kit of parts according to clause 181, wherein the pharmaceutical composition comprising the hyaluronidase is a solution, and the concentration of the hyaluronidase in the solution is from about 50 to about 10,000 U/mL, in particular is about 2,000 U/mL.
183. The kit of parts according to any one of clauses 169-179, wherein the rilpivirine or a pharmaceutically acceptable salt thereof and the hyaluronidase are formulated for administration as a combined pharmaceutical composition.
184. The kit of parts according to any one of clauses 169-183, wherein the rilpivirine or a pharmaceutically acceptable salt thereof and the hyaluronidase are formulated for administration by subcutaneous injection.
185. The kit of parts according to any one of clauses 169-184, wherein the suspension comprises a pharmaceutically acceptable aqueous carrier in which the rilpivirine or a pharmaceutically acceptable salt thereof is suspended.
186. The kit of parts according to any one of clauses 169-185, wherein the rilpivirine or a pharmaceutically acceptable salt thereof is rilpivirine.
187. The rilpivirine or a pharmaceutically acceptable salt thereof and a hyaluronidase for use according to any one of clauses 1-9 and 15-25 when not dependent on any one of clauses 10-14, wherein the micro- or nanoparticles have an average effective particle size of from about 0.2 μm to about 3 μm.
188. The rilpivirine or a pharmaceutically acceptable salt thereof and a hyaluronidase for use according to clause 187, wherein the micro- or nanoparticles have an average effective particle size of from about 1 μm to about 3 μm, preferably about 1.5 μm to about 3 μm, more preferably about 2 μm to about 3 μm.
189. The rilpivirine or a pharmaceutically acceptable salt thereof and a hyaluronidase for use according to clause 187, wherein the micro- or nanoparticles have an average effective particle size of from about 1 μm to about 2.5 μm.
190. The rilpivirine or a pharmaceutically acceptable salt thereof and a hyaluronidase for use according to clause 189, wherein the micro- or nanoparticles have an average effective particle size of about 2.5 μm.
191. The rilpivirine or a pharmaceutically acceptable salt thereof and a hyaluronidase for use according to any one of clauses 1-9 and 15-25 when not dependent on any one of clauses 10-14, wherein the micro- or nanoparticles have a Dᵥ90 of from about 2 μm to about 7 μm.
192. The rilpivirine or a pharmaceutically acceptable salt thereof and a hyaluronidase for use according to clause 191, wherein the micro- or nanoparticles have a Dᵥ90 of from about 3 μm to about 6 μm.
193. The rilpivirine or a pharmaceutically acceptable salt thereof and a hyaluronidase for use according to clause 192, wherein the micro- or nanoparticles have a Dᵥ90 of from about 3 μm to about 5.5 μm.
194. The rilpivirine or a pharmaceutically acceptable salt thereof and a hyaluronidase for use according to clause 193, wherein the micro- or nanoparticles have a Dᵥ90 of about 5.5 μm.
195. The rilpivirine or a pharmaceutically acceptable salt thereof and a hyaluronidase for use according to any one of clauses 1-9 and 15-25 when not dependent on any one of clauses 10-14, wherein the micro- or nanoparticles have an average effective particle size of from about 0.2 μm to about 3 μm and a Dᵥ90 of from about 1.8 μm to about 7 μm.
196. The combination for use according to any one of clauses 26-34 and 40-50 when not dependent on any one of clauses 35-39, wherein the micro- or nanoparticles have an average effective particle size of from about 0.2 μm to about 3 μm.
197. The combination for use according to clause 196, wherein the micro- or nanoparticles have an average effective particle size of from about 1 μm to about 3 μm, preferably about 1.5 μm to about 3 μm, more preferably about 2 μm to about 3 μm.
198. The combination for use according to clause 196, wherein the micro- or nanoparticles have an average effective particle size of from about 1 μm to about 2.5 μm.
199. The combination for use according to clause 198, wherein the micro- or nanoparticles have an average effective particle size of about 2.5 μm.
200. The combination for use according to any one of clauses 26-34 and 40-50 when not dependent on any one of clauses 35-39, wherein the micro- or nanoparticles have a Dᵥ90 of from about 2 μm to about 7 μm.
201. The combination for use according to clause 200, wherein the micro- or nanoparticles have a Dᵥ90 of from about 3 μm to about 6 μm.
202. The combination for use according to clause 201, wherein the micro- or nanoparticles have a Dᵥ90 of from about 3 μm to about 5.5 μm.
203. The combination for use according to clause 202, wherein the micro- or nanoparticles have a Dᵥ90 of about 5.5 μm.
204. The combination for use according to any one of clauses 26-34 and 40-50 when not dependent on any one of clauses 35-39, wherein the micro- or nanoparticles have an average effective particle size of from about 0.2 μm to about 3 μm and a Dᵥ90 of from about 1.8 μm to about 7 μm.
205. The products for simultaneous or sequential use according to any one of clauses 51-59 and 65-75 when not dependent on any one of clauses 60-64, wherein the micro- or nanoparticles have an average effective particle size of from about 0.2 μm to about 3 μm.
206. The products for simultaneous or sequential use according to clause 205, wherein the micro- or nanoparticles have an average effective particle size of from about 1 μm to about 3 μm, preferably about 1.5 μm to about 3 μm, more preferably about 2 μm to about 3 μm.
207. The products for simultaneous or sequential use according to clause 205, wherein the micro- or nanoparticles have an average effective particle size of from about 1 μm to about 2.5 μm.
208. The products for simultaneous or sequential use according to clause 207, wherein the micro- or nanoparticles have an average effective particle size of about 2.5 μm.
209. The products for simultaneous or sequential use according to any one of clauses 51-59 and 65-75 when not dependent on any one of clauses 60-64, wherein the micro- or nanoparticles have a Dᵥ90 of from about 2 μm to about 7 μm.
210. The products for simultaneous or sequential use according to clause 209, wherein the micro- or nanoparticles have a Dᵥ90 of from about 3 μm to about 6 μm.
211. The products for simultaneous or sequential use according to clause 210, wherein the micro- or nanoparticles have a Dᵥ90 of from about 3 μm to about 5.5 μm.
212. The products for simultaneous or sequential use according to clause 211, wherein the micro- or nanoparticles have a Dᵥ90 of about 5.5 μm.
213. The products for simultaneous or sequential use according to any one of clauses 51-59 and 65-75 when not dependent on any one of clauses 60-64, wherein the micro- or nanoparticles have an average effective particle size of from about 0.2 μm to about 3 μm and a Dᵥ90 of from about 1.8 μm to about 7 μm.
214. The kit of parts for simultaneous or sequential use according to any one of clauses 76-84 and 90-100 when not dependent on any one of clauses 85-89, wherein the micro- or nanoparticles have an average effective particle size of from about 0.2 μm to about 3 μm.
215. The kit of parts for simultaneous or sequential use according to clause 214, wherein the micro- or nanoparticles have an average effective particle size of from about 1 μm to about 3 μm, preferably about 1.5 μm to about 3 μm, more preferably about 2 μm to about 3 μm.
216. The kit of parts for simultaneous or sequential use according to clause 214, wherein the micro- or nanoparticles have an average effective particle size of from about 1 μm to about 2.5 μm.
217. The kit of parts for simultaneous or sequential use according to clause 216, wherein the micro- or nanoparticles have an average effective particle size of about 2.5 μm.
218. The kit of parts for simultaneous or sequential use according to any one of clauses 76-84 and 90-100 when not dependent on any one of clauses 85-89, wherein the micro- or nanoparticles have a Dᵥ90 of from about 2 μm to about 7 μm.
219. The kit of parts for simultaneous or sequential use according to clause 218, wherein the micro- or nanoparticles have a Dᵥ90 of from about 3 μm to about 6 μm.
220. The kit of parts for simultaneous or sequential use according to clause 219, wherein the micro- or nanoparticles have a Dᵥ90 of from about 3 μm to about 5.5 μm.
221. The kit of parts for simultaneous or sequential use according to clause 210, wherein the micro- or nanoparticles have a Dᵥ90 of about 5.5 μm.
222. The kit of parts for simultaneous or sequential use according to any one of clauses 76-84 and 90-100 when not dependent on any one of clauses 85-89, wherein the micro- or nanoparticles have an average effective particle size of from about 0.2 μm to about 3 μm and a Dᵥ90 of from about 1.8 μm to about 7 μm.
223. The rilpivirine or a pharmaceutically acceptable salt thereof for use according to any one of clauses 101-109 and 115-125 when not dependent on any one of clauses 110-114, wherein the micro- or nanoparticles have an average effective particle size of from about 0.2 μm to about 3 μm.
224. The rilpivirine or a pharmaceutically acceptable salt thereof for use according to clause 223, wherein the micro- or nanoparticles have an average effective particle size of from about 1 μm to about 3 μm, preferably about 1.5 μm to about 3 μm, more preferably about 2 μm to about 3 μm.
225. The rilpivirine or a pharmaceutically acceptable salt thereof for use according to clause 223, wherein the micro- or nanoparticles have an average effective particle size of from about 1 μm to about 2.5 μm.
226. The rilpivirine or a pharmaceutically acceptable salt thereof for use according to clause 225, wherein the micro- or nanoparticles have an average effective particle size of about 2.5 μm.
227. The rilpivirine or a pharmaceutically acceptable salt thereof for use according to any one of clauses 101-109 and 115-125 when not dependent on any one of clauses 110-114, wherein the micro- or nanoparticles have a Dᵥ90 of from about 2 μm to about 7 μm.
228. The rilpivirine or a pharmaceutically acceptable salt thereof for use according to clause 227, wherein the micro- or nanoparticles have a Dᵥ90 of from about 3 μm to about 6 μm.
229. The rilpivirine or a pharmaceutically acceptable salt thereof for use according to clause 228, wherein the micro- or nanoparticles have a Dᵥ90 of from about 3 μm to about 230. The rilpivirine or a pharmaceutically acceptable salt thereof for use according to clause 229, wherein the micro- or nanoparticles have a Dᵥ90 of about 5.5 μm.
231. The rilpivirine or a pharmaceutically acceptable salt thereof for use according to any one of clauses 101-109 and 115-125 when not dependent on any one of clauses 110-114, wherein the micro- or nanoparticles have an average effective particle size of from about 0.2 μm to about 3 μm and a Dᵥ90 of from about 1.8 μm to about 7 μm.
232. The use according to any one of clauses 126-134 and 140-150 when not dependent on any one of clauses 135-139, wherein the micro- or nanoparticles have an average effective particle size of from about 0.2 μm to about 3 μm.
233. The use according to clauses 232, wherein the micro- or nanoparticles have an average effective particle size of from about 1 μm to about 3 μm, preferably about 1.5 μm to about 3 μm, more preferably about 2 μm to about 3 μm.
234. The use according to clause 232, wherein the micro- or nanoparticles have an average effective particle size of from about 1 μm to about 2.5 μm.
235. The use according to clause 234, wherein the micro- or nanoparticles have an average effective particle size of about 2.5 μm.
236. The use according to any one of clauses 126-134 and 140-150 when not dependent on any one of clauses 135-139, wherein the micro- or nanoparticles have a Dᵥ90 of from about 2 μm to about 7 μm.
237. The use according to clauses 236, wherein the micro- or nanoparticles have a Dᵥ90 of from about 3 μm to about 6 μm.
238. The use according to clause 237, wherein the micro- or nanoparticles have a Dᵥ90 of from about 3 μm to about 5.5 μm.
239. The use according to clause 238, wherein the micro- or nanoparticles have a Dᵥ90 of about 5.5 μm.
240. The use according to any one of clauses 126-134 and 140-150 when not dependent on any one of clauses 135-139, wherein the micro- or nanoparticles have an average effective particle size of from about 0.2 μm to about 3 μm and a Dᵥ90 of from about 1.8 μm to about 7 μm.
241. The combination according to any one of clauses 151-156 and 162-169 when not dependent on any one of clauses 157-161, wherein the micro- or nanoparticles have an average effective particle size of from about 0.2 μm to about 3 μm.
242. The combination according to clause 241, wherein the micro- or nanoparticles have an average effective particle size of from about 1 μm to about 3 μm, preferably about 1.5 μm to about 3 μm, more preferably about 2 μm to about 3 μm.
243. The combination according to clause 241, wherein the micro- or nanoparticles have an average effective particle size of from about 1 μm to about 2.5 μm.
244. The combination according to clause 243, wherein the micro- or nanoparticles have an average effective particle size of about 2.5 μm.
245. The combination according to any one of clauses 151-156 and 162-168 when not dependent on any one of clauses 157-161, wherein the micro- or nanoparticles have a Dᵥ90 of from about 2 μm to about 7 μm.
246. The combination according to clause 245, wherein the micro- or nanoparticles have a Dᵥ90 of from about 3 μm to about 6 μm.
247. The combination according to clause 246, wherein the micro- or nanoparticles have a Dᵥ90 of from about 3 μm to about 5.5 μm.
248. The combination according to clause 247, wherein the micro- or nanoparticles have a Dᵥ90 of about 5.5 μm.
249. The combination according to any one of clauses 151-156 and 162-168 when not dependent on any one of clauses 157-161, wherein the micro- or nanoparticles have an average effective particle size of from about 0.2 μm to about 3 μm and a Dᵥ90 of from about 1.8 μm to about 7 μm.
250. The kit of parts according to any one of clauses 169-174 and 180-186 when not dependent on any one of clauses 175-179, wherein the micro- or nanoparticles have an average effective particle size of from about 0.2 μm to about 3 μm.
251. The kit of parts according to clause 250, wherein the micro- or nanoparticles have an average effective particle size of from about 1 μm to about 3 μm, preferably about 1.5 μm to about 3 μm, more preferably about 2 μm to about 3 μm.
252. The kit of parts according to clause 250, wherein the micro- or nanoparticles have an average effective particle size of from about 1 μm to about 2.5 μm.
253. The kit of parts according to clause 252, wherein the micro- or nanoparticles have an average effective article size of about 2.5 μm.
254. The kit of parts according to any one of clauses 169-174 and 180-86 when not dependent on any one of clauses 175-179, wherein the micro- or nanoparticles have a Dᵥ90 of from about 2 μm to about 7 μm.
255. The kit of parts according to clause 254, wherein the micro- or nanoparticles have a Dᵥ90 of from about 3 μm to about 6 μm.
256. The kit of parts according to clause 255, wherein the micro- or nanoparticles have a Dᵥ90 of from about 3 μm to about 5.5 μm.
257. The kit of parts according to clause 256, wherein the micro- or nanoparticles have a Dᵥ90 of about 5.5 μm.
258. The kit of parts according to any one of clauses 169-174 and 180-186 when not dependent on any one of clauses 175-179, wherein the micro- or nanoparticles have an average effective particle size of from about 0.2 μm to about 3 μm and a Dᵥ90 of from about 1.8 μm to about 7 μm.
259. Rilpivirine or a pharmaceutically acceptable salt thereof in the form of micro- or nanoparticles in suspension, wherein the micro- or nanoparticles have a Dᵥ90 of from about 1 μm to about 10 μm.
260. The rilpivirine or a pharmaceutically acceptable salt thereof according to clause 259, wherein the micro- or nanoparticles have a Dᵥ90 of from about 1 μm to about 7 μm, preferably from about 2 μm to about 7 μm.
261. The rilpivirine or a pharmaceutically acceptable salt thereof according to clause 260, wherein the micro- or nanoparticles have a Dᵥ90 of from about 3 μm to about 6 μm.
262. The rilpivirine or a pharmaceutically acceptable salt thereof according to clause 261, wherein the micro- or nanoparticles have a Dᵥ90 of from about 3 μm to about 5.5 μm.
263. The rilpivirine or a pharmaceutically acceptable salt thereof according to clause 259, wherein the micro- or nanoparticles have a Dᵥ90 of from about 1.8 μm to about 7 μm and an average effective particle size (Dᵥ50) of from about 0.2 μm to about 3 μm.
264. The rilpivirine or a pharmaceutically acceptable salt thereof according to clause 262 or clause 263, wherein the Dᵥ90 is about 5.5 μm.
265. The rilpivirine or a pharmaceutically acceptable salt thereof according to clause 263 or clause 264, wherein the average effective particle size is about 2.5 μm.
266. The rilpivirine or a pharmaceutically acceptable salt thereof according to any one of clauses 259-265, wherein the micro- or nanoparticles have a surface modifier adsorbed to their surface.
267. The rilpivirine or a pharmaceutically acceptable salt thereof according to clause 266, wherein the surface modifier is a poloxamer.
268. The rilpivirine or a pharmaceutically acceptable salt thereof according to clause 267, wherein the poloxamer is poloxamer 338.
269. The rilpivirine or a pharmaceutically acceptable salt thereof according to any one of clauses 259-268, wherein the suspension comprises a pharmaceutically acceptable aqueous carrier in which the rilpivirine or a pharmaceutically acceptable salt thereof is suspended.
270. The rilpivirine or a pharmaceutically acceptable salt thereof according to any one of clauses 259-269, wherein the rilpivirine or a pharmaceutically acceptable salt thereof is rilpivirine (i.e. rilpivirine free base).
271. A pharmaceutical composition comprising rilpivirine or a pharmaceutically acceptable salt thereof in the form of micro- or nanoparticles in suspension as defined in any one of clauses 259-270.
272. The pharmaceutical composition according to clause 271, wherein pharmaceutical composition is formulated for administration by subcutaneous or intramuscular injection.
273. The pharmaceutical composition according to clause 271, wherein the pharmaceutical composition is formulated for administration by subcutaneous injection.
274. Rilpivirine or a pharmaceutically acceptable salt thereof as defined in any one of clauses 259-270, for use in the treatment or prevention of HIV infection in a subject.
275. A method for treating or preventing HIV infection in a subject, the method comprising administering rilpivirine or a pharmaceutically acceptable salt thereof as defined in any one of clauses 259-270 to the subject.
276. Use of rilpivirine or a pharmaceutically acceptable salt thereof as defined in any one of clauses 259-270 for the manufacture of a medicament for treating or preventing HIV infection in a subject.
277. The rilpivirine or a pharmaceutically acceptable salt thereof for use according to clause 274, method according to clause 275 or use according to clause 276, wherein the rilpivirine or pharmaceutically acceptable salt thereof is administered to the subject at a time interval of about three months to about two years.
278. The rilpivirine or a pharmaceutically acceptable salt thereof for use, method or use according to clause 277, wherein the time interval is about three months to about six months.
279. The rilpivirine or a pharmaceutically acceptable salt thereof for use, method or use according to clause 277, wherein the time interval is about six months to about two years.
280. The rilpivirine or a pharmaceutically acceptable salt thereof for use, method or use according to clause 279, wherein the time interval is about six months to about one year, in particular is about 6 months.
281. The rilpivirine or a pharmaceutically acceptable salt thereof for use, method or use according to any one of clauses 274-280, wherein the rilpivirine or a pharmaceutically acceptable salt thereof is administered to the subject by subcutaneous or intramuscular injection.
282. The rilpivirine or a pharmaceutically acceptable salt thereof for use, method or use according to clause 281, wherein the rilpivirine or a pharmaceutically acceptable salt thereof is administered to the subject by subcutaneous injection.
283. The rilpivirine or a pharmaceutically acceptable salt thereof for use, method or use according to any one of clauses 274-282, wherein the treatment or prevention of HIV infection is treatment of HIV infection.
284. The rilpivirine or a pharmaceutically acceptable salt thereof for use, method or use according to clause 283, wherein each administration of the rilpivirine or a pharmaceutically acceptable salt thereof comprises from about 2700 mg to about 5400 mg of rilpivirine or a pharmaceutically acceptable salt thereof.
285. The rilpivirine or a pharmaceutically acceptable salt thereof for use, method or use according to any one of clauses 274-284, wherein the HIV infection is HIV type 1 (HIV-1) infection.
286. The rilpivirine or a pharmaceutically acceptable salt thereof for use, method or use according to any one of clauses 274-285, wherein the subject is a human.

1. A method for the treatment or prevention of HIV infection in a subject in need thereof, the method comprising administering to the subject a therapeutically effective amount of rilpivirine or a pharmaceutically acceptable salt thereof in the form of micro- or nanoparticles in suspension by intramuscular injection or subcutaneous injection,
wherein the rilpivirine or a pharmaceutically acceptable salt thereof is administered in combination with a hyaluronidase that is administered by intramuscular injection or subcutaneous injection, and
wherein the rilpivirine or a pharmaceutically acceptable salt thereof and the hyaluronidase are administered intermittently at a time interval of about three months to about two years.

2. The method according to claim 1, wherein the hyaluronidase is recombinant human hyaluronidase (e.g. rHuPH20), for example, comprising the amino acid sequence of SEQ ID NO: 1.

3. The method according to any one of the preceding claims, wherein the time interval is about three months to about one year.

4. The method according to claim 3, wherein the time interval is about three months to about six months.

5. The method according to claim 3, wherein the time interval is about six months to about one year, preferably wherein the time interval is about six months.

6. The method according to any one of the preceding claims, wherein the rilpivirine or a pharmaceutically acceptable salt thereof and hyaluronidase are administered simultaneously or sequentially.

7. The method according to any one of the preceding claims, wherein the micro- or nanoparticles have a surface modifier adsorbed to their surface.

8. The method according to claim 7, wherein the surface modifier is a poloxamer.

9. The method according to claim 8, wherein the poloxamer is poloxamer 338.

10. The method according to any one of the preceding claims, wherein the average effective particle size of the micro- or nanoparticles is less than about 1 μm.

11. The method according to claim 10, wherein the average effective particle size of the micro- or nanoparticles is less than about 500 nm.

12. The method according to claim 11, wherein the average effective particle size of the micro- or nanoparticles is from about 100 nm to about 300 nm.

13. The method according to claim 12, wherein the average effective particle size of the micro- or nanoparticles is from about 150 nm to about 250 nm.

14. The method according to claim 13, wherein the average effective particle size of the micro- or nanoparticles is from about 180 nm to about 220 nm.

15. The method according to any one of claims 1-9, wherein the micro- or nanoparticles have an average effective particle size of from about 0.2 μm to about 3 μm.

16. The method according to claim 15, wherein the micro- or nanoparticles have an average effective particle size of from about 1 μm to about 3 μm, preferably about 1.5 μm to about 3 μm, more preferably about 2 μm to about 3 μm.

17. The method according to claim 15, wherein the micro- or nanoparticles have an average effective particle size of from about 1 μm to about 2.5 μm.

18. The method according to claim 17, wherein the micro- or nanoparticles have an average effective particle size of about 2.5 μm.

19. The method according to any one of clauses 1-9, wherein the micro- or nanoparticles have a Dᵥ90 of from about 2 μm to about 7 μm.

20. The method according to claim 19, wherein the micro- or nanoparticles have a Dᵥ90 of from about 3 μm to about 6 μm.

21. The method according to claim 20, wherein the micro- or nanoparticles have a Dᵥ90 of from about 3 μm to about 5.5 μm.

22. The method according to claim 21, wherein the micro- or nanoparticles have a Dᵥ90 of about 5.5 μm.

23. The method according to any one of the preceding claims, wherein the rilpivirine or a pharmaceutically acceptable salt thereof and the hyaluronidase are administered sequentially.

24. The method according to any one of the preceding claims, wherein the rilpivirine or a pharmaceutically acceptable salt thereof and hyaluronidase are administered in separate pharmaceutical compositions.

25. The method according to claim 24, wherein the pharmaceutical composition comprising the hyaluronidase is a solution, and the concentration of the hyaluronidase in the solution is from about 50 to about 10,000 U/mL, preferably about 2,000 U/mL.

26. The method according to any one of claims 1-22, wherein the rilpivirine or a pharmaceutically acceptable salt thereof and the hyaluronidase are administered as a combined pharmaceutical composition.

27. The method according to any one of the preceding claims, wherein the rilpivirine or a pharmaceutically acceptable salt thereof and the hyaluronidase are administered by subcutaneous injection.

28. The method according to any one of the preceding claims, wherein the suspension comprises a pharmaceutically acceptable aqueous carrier in which the rilpivirine or a pharmaceutically acceptable salt thereof is suspended.

29. The method according to any one of the preceding claims, wherein the method is a method of treatment of HIV infection.

30. The method according to claim 29, wherein each administration of the rilpivirine or a pharmaceutically acceptable salt thereof comprises from about 2700 mg to about 5400 mg of rilpivirine or a pharmaceutically acceptable salt thereof, preferably from about 2700 mg to about 4500 mg of rilpivirine or a pharmaceutically acceptable salt thereof.

31. The method according to any one of the preceding claims, wherein the HIV infection is HIV type 1 (HIV-1) infection.

32. The method according to any one of the preceding claims, wherein the subject is a human.

33. The method according to any one of the preceding claims, wherein the rilpivirine or a pharmaceutically acceptable salt thereof is rilpivirine.

34. Rilpivirine or a pharmaceutically acceptable salt thereof and a hyaluronidase for use in the treatment or prevention of HIV infection in a subject,
wherein the rilpivirine or a pharmaceutically acceptable salt thereof is in the form of micro- or nanoparticles in suspension,
wherein the rilpivirine or pharmaceutically acceptable salt thereof and hyaluronidase are administered to the subject by intramuscular injection or subcutaneous injection, and
wherein the rilpivirine or pharmaceutically acceptable salt thereof and hyaluronidase are administered intermittently at a time interval of about three months to about two years.

35. Products containing rilpivirine or a pharmaceutically acceptable salt thereof and a hyaluronidase as a combined preparation for simultaneous or sequential use in the treatment or prevention of HIV infection by intramuscular injection or subcutaneous injection,
wherein the rilpivirine or pharmaceutically acceptable salt thereof is in the form of micro- or nanoparticles in suspension, and
wherein the rilpivirine or pharmaceutically acceptable salt thereof and the hyaluronidase are administered intermittently at a time interval of about three months to about two years.

36. A kit of parts comprising rilpivirine or a pharmaceutically acceptable salt thereof and a hyaluronidase for simultaneous or sequential use in the treatment or prevention of HIV infection by intramuscular injection or subcutaneous injection,
wherein the rilpivirine or a pharmaceutically acceptable salt thereof is in the form of micro- or nanoparticles in suspension, and
wherein the rilpivirine or a pharmaceutically acceptable salt thereof and the hyaluronidase are administered intermittently at a time interval of about three months to about two years.

37. Rilpivirine or a pharmaceutically acceptable salt thereof in the form of micro- or nanoparticles in suspension for use in the treatment or prevention of HIV infection by intramuscular injection or subcutaneous injection,
wherein the rilpivirine or pharmaceutically acceptable salt thereof is administered in combination with a hyaluronidase that is administered by intramuscular injection or subcutaneous injection, and
wherein the rilpivirine or pharmaceutically acceptable salt thereof and the hyaluronidase are administered intermittently at a time interval of about three months to about two years.

38. Use of rilpivirine or a pharmaceutically acceptable salt thereof for the manufacture of a medicament for treating or preventing HIV infection in a subject,
wherein the rilpivirine or pharmaceutically acceptable salt thereof is in the form of micro- or nanoparticles in suspension and is administered in combination with a hyaluronidase,
wherein the rilpivirine or pharmaceutically acceptable salt thereof and the hyaluronidase are administered to the subject by intramuscular injection or subcutaneous injection, and
wherein the rilpivirine or pharmaceutically acceptable salt thereof and the hyaluronidase are administered intermittently at a time interval of about three months to about two years.

39. A combination comprising rilpivirine or a pharmaceutically acceptable salt thereof and a hyaluronidase, wherein the rilpivirine or a pharmaceutically acceptable salt thereof is in the form of micro- or nanoparticles in suspension.

40. A kit of parts comprising rilpivirine or a pharmaceutically acceptable salt thereof and a hyaluronidase, wherein the rilpivirine or a pharmaceutically acceptable salt thereof is in the form of micro- or nanoparticles in suspension.

41. Rilpivirine or a pharmaceutically acceptable salt thereof in the form of micro- or nanoparticles in suspension, wherein the micro- or nanoparticles have a Dᵥ90 of from about 1 μm to about 10 μm.

42. The rilpivirine or a pharmaceutically acceptable salt thereof according to claim 41, wherein the micro- or nanoparticles have a Dᵥ90 of from about 1 μm to about 7 μm, preferably from about 2 μm to about 7 μm.

43. The rilpivirine or a pharmaceutically acceptable salt thereof according to claim 42, wherein the micro- or nanoparticles have a Dᵥ90 of from about 3 μm to about 6 μm, preferably from about 3 μm to about 5.5 μm.

44. The rilpivirine or a pharmaceutically acceptable salt thereof according to claim 41, wherein the micro- or nanoparticles have a Dᵥ90 of from about 1.8 μm to about 7 μm and an average effective particle size of from about 0.2 μm to about 3 μm.

45. The rilpivirine or a pharmaceutically acceptable salt thereof according to claim 43 or claim 44, wherein the Dᵥ90 is about 5.5 μm.

46. The rilpivirine or a pharmaceutically acceptable salt thereof according to claim 44 or claim 45, wherein the average effective particle size is about 2.5 μm.

47. The rilpivirine or a pharmaceutically acceptable salt thereof according to any one of claims 41-46, wherein the micro- or nanoparticles have a surface modifier adsorbed to their surface.

48. The rilpivirine or a pharmaceutically acceptable salt thereof according to claim 47, wherein the surface modifier is a poloxamer.

49. The rilpivirine or a pharmaceutically acceptable salt thereof according to claim 48, wherein the poloxamer is poloxamer 338.

50. The rilpivirine or a pharmaceutically acceptable salt thereof according to any one of claims 41-49, wherein the suspension comprises a pharmaceutically acceptable aqueous carrier in which the rilpivirine or a pharmaceutically acceptable salt thereof is suspended.

51. The rilpivirine or a pharmaceutically acceptable salt thereof according to any one of claims 41-50, wherein the rilpivirine or a pharmaceutically acceptable salt thereof is rilpivirine.

52. A pharmaceutical composition comprising rilpivirine or a pharmaceutically acceptable salt thereof in the form of micro- or nanoparticles in suspension as defined in any one of claims 41-51.

53. The pharmaceutical composition according to claim 52, wherein pharmaceutical composition is formulated for administration by subcutaneous or intramuscular injection.

54. The pharmaceutical composition according to claim 53, wherein the pharmaceutical composition is formulated for administration by subcutaneous injection.

55. Rilpivirine or a pharmaceutically acceptable salt thereof as defined in any one of claims 41-51, for use in the treatment or prevention of HIV infection in a subject.

56. A method for treating or preventing HIV infection in a subject, the method comprising administering rilpivirine or a pharmaceutically acceptable salt thereof as defined in any one of claims 41-51 to the subject.

57. Use of rilpivirine or a pharmaceutically acceptable salt thereof as defined in any one of claims 41-51 for the manufacture of a medicament for treating or preventing HIV infection in a subject.

58. The rilpivirine or a pharmaceutically acceptable salt thereof for use according to claim method according to claim 56 or use according to claim 57, wherein the rilpivirine or a pharmaceutically acceptable salt thereof is administered to the subject at a time interval of about three months to about two years.

59. The rilpivirine or a pharmaceutically acceptable salt thereof for use, method or use according to claim 58, wherein the time interval is about three months to about six months.

60. The rilpivirine or a pharmaceutically acceptable salt thereof for use, method or use according to claim 58, wherein the time interval is about six months to about one year, preferably wherein the time interval is about 6 months.

61. The rilpivirine or a pharmaceutically acceptable salt thereof for use, method or use according to any one of claims 55-60, wherein the rilpivirine or a pharmaceutically acceptable salt thereof is administered to the subject by subcutaneous or intramuscular injection.

62. The rilpivirine or a pharmaceutically acceptable salt thereof for use, method or use according to claim 61, wherein the rilpivirine or a pharmaceutically acceptable salt thereof is administered to the subject by subcutaneous injection.

63. The rilpivirine or a pharmaceutically acceptable salt thereof for use, method or use according to any one of claims 55-62, wherein the treatment or prevention of HIV infection is treatment of HIV infection.

64. The rilpivirine or a pharmaceutically acceptable salt thereof for use, method or use according to claim 63, wherein each administration of the rilpivirine or a pharmaceutically acceptable salt thereof comprises from about 2700 mg to about 5400 mg of rilpivirine or a pharmaceutically acceptable salt thereof, preferably from about 2700 mg to about 4500 mg of rilpivirine or a pharmaceutically acceptable salt thereof.

65. The rilpivirine or a pharmaceutically acceptable salt thereof for use, method or use according to any one of claims 55-64, wherein the HIV infection is HIV type 1 (HIV-1) infection.

66. The rilpivirine or a pharmaceutically acceptable salt thereof for use, method or use according to any one of claims 55-65, wherein the subject is a human.


SEQUENCE LISTING (5 sequences)

SEQ ID NO: 1 - protein, 443 aa, Artificial Sequence
  rHuPH20
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 2 - protein, 447 aa, Artificial Sequence [sequence omitted]

SEQ ID NO: 3 - protein, 446 aa, Artificial Sequence
  rHuPH20 variant 2
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 4 - protein, 445 aa, Artificial Sequence
  rHuPH20 variant 3
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 5 - protein, 444 aa, Artificial Sequence
  rHuPH20 variant 4
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ